(19) United States
(12) Patent Application Publication
(10) Pub. No.: US 2012/0225417 A1
(43) Pub. Date: Sep. 6, 2012

(54) Methods, Compositions and Kits for the Evaluation and Preservation of Sperm

(75) Inventors: François Richard, Quebec (CA); Christine Guillemette, Quebec (CA); Juan Pablo Aragon, Quebec (CA); Audrey Hébert, Quebec (CA); Pierre Leclerc, St-Nicolas (CA); Patrick Blondin, Ste-Julie (CA)

(73) Assignees: I'Alliance Boviteq Inc., Saint-Hyacinthe (CA); Université Laval, Quebec (CA)

(21) Appl. No.: 13/365,838

(22) Filed: Feb. 3, 2012

Related U.S. Application Data
(60) Provisional application No. 61/441,003, filed on Feb. 9, 2011.

(51) Int. Cl. C12N 5/076; C12Q 1/44

(52) U.S. Cl. 435/2; 435/19

(57) ABSTRACT

The invention concerns methods and kits for evaluating the semen quality. The invention also concerns compounds and compositions for preserving a sperm sample, especially during cryopreservation. The invention is based on a correlation between sperm phosphodiesterase (PDE) activity and sperm fertility and on the use of selective phosphodiesterase inhibitors, more particularly PDE10 inhibitors.

### FIELD OF THE INVENTION

The present invention relates to the field of reproductive biology, and particularly to mammalian semen for use in insemination. More specifically, the present invention is concerned with methods and kits for evaluating the quality of semen. The invention is also concerned with compounds and compositions for preserving a sperm sample, especially during cryopreservation.

### BACKGROUND OF THE INVENTION

Semen cryopreservation is largely used in bovine and human. A great advantage of cryopreservation is the ability to preserve for many years a given ejaculate for later use in insemination or in vitro fertilization (IVF). However, the quality of the semen is largely affected during the freeze-thaw process and it is still not clear whether the damages are due to the process itself, the extenders or diluents in which the spermatozoa are suspended, or by the combination of those or other factors.

Great deals of efforts are being invested at improving semen cryopreservation and developing new semen extenders. Different approaches have been considered, including prevention of sperm DNA damage, preservation of sperm membrane integrity, elimination of harmful reactive oxygen species, etc. but none have considered the significance of cyclic nucleotide phosphodiesterases (PDEs). Cyclic nucleotide phosphodiesterases catalyze the hydrolysis of cyclic nucleotides such as cAMP and cGMP, and the PDEs thus play critical regulatory roles in a wide variety of signal transduction pathways. So far, eleven PDE families have been recognized in mammalian tissues (see Menniti et al. Nature Reviews Drug Discovery (2006); 5:660-670), including human sperm (Richter et al., Mol Hum Reprod (1999), 5: 732-6) and mouse sperm (Baxendale and Fraser. Mol Reprod Dev (2005), 71: 495-508).

PDE inhibitors have long been used for studying sperm-specific processes such as motility, capacitation, agglutination, acrosome reaction and hyperactivation. Selected examples of known selective and non-selective PDE inhibitors used for studying sperms include pentoxifylline, rolipram, IBMX, and papaverine. However, no one has ever suggested the use of selective PDE10 inhibitors for the preservation of sperm functions during one or more of the steps of manipulation, preparation, dilution, freezing, thawing, cell-sorting, and/or sex-sorting of spermatozoa to which the spermatozoa may be subjected, especially during in vitro fertilization, artificial insemination and/or cryopreservation.

PDE10 is one of the most recent PDE family identified. Cloning of the mouse PDE10A has been published in patent application U.S. 2006/166316. Although this enzyme has been reported to have some roles in spermatozoa physiology (Coskran et al., J Histochem Cytochem (2006), 54: 1205-13; Wayman et al., Int J Impot Res (2005), 17:216-23), no one has ever suggested a correlation between the enzymatic activity of PDE10 with the quality of semen, let alone the future sperm performance (e.g. fertility) after cryopreservation.

In view of the above, there is thus a need for improved semen extenders, including semen extenders for use during insemination with liquid phase semen and extenders for use during semen cryopreservation. There is also a need for methods and kits for assessing the quality of the semen before, during, and/or after the many steps of manipulation, preparation, dilution, freezing, thawing, cell-sorting, and/or sex-sorting in which the spermatozoa may be subjected, especially during in vitro fertilization, artificial insemination and/or cryopreservation processes.

### SUMMARY OF THE INVENTION

The present invention provides methods for evaluating a mammalian sperm sample by measuring sperm phosphodiesterase (PDE) enzymatic activity in the sample. In preferred embodiment, the PDE enzymatic activity is measured in isolated spermatozoa.

The present invention also relates to the use of phosphodiesterase (PDE) inhibitor(s) for preserving a sperm sample, particularly before, during, and/or after the many steps of manipulation, preparation, dilution, freezing, thawing, cell-sorting, and/or sex-sorting in which the spermatozoa may be subjected, especially during in vitro fertilization, artificial insemination and/or cryopreservation processes. In a related aspect, the invention provides a semen extender for preserving a sperm sample, the extender comprising a phosphodiesterase (PDE) inhibitor.

In preferred embodiments the PDE inhibitor is PDE10 specific. More preferably, the PDE inhibitor is cAMP papaverine-sensitive. Even more preferably, the PDE inhibitor is papaverine. According to more specific aspects, the present invention pertains to the use of papaverine in semen extenders for preserving a sperm sample during the cryopreservation process (i.e. freeze-thaw).

According to a further aspect, the invention relates to a commercial package or kit for measuring PDE10 activity in a mammalian sperm sample or for preserving mammalian sperm.

An advantage of the present invention is that it provides effective means for promptly evaluating and/or predicting sperm quality, integrity and/or fertility. The invention also provide means for improving preservation sperm dedicated to artificial insemination, and more particularly means for preserving sperm integrity and fertility during the cryopreservation process.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE DRAWINGS

FIG. 1 is panel showing the expression of PDE10A as determined by RT-PCR of extracts from Bovine Testis, Caput and Cauda of epididymis and seminal vesicles. Total RNA was isolated from bovine testis (TS), caput (CT), cauda (CD) and seminal vesicles (SV) and was reverse transcribed into cDNA. 1 ug of cDNA for each tissue was subjected to PCR reactions using two 502 bp specific primers designed according the predicted Bos taurus phosphodiesterase 10A (PDE10A, amplifying from 1730 to 2231 of sequence defined at Accession number: XM_{—}582454). As seen PDE10A transcripts are present in all tissues analyzed at the expected height. The primer alone (−) was loaded as a negative control. The homology of the PCR products to the predicted Bos taurus phosphodiesterase 10A (XM_{—}582454) are reported and are higher than 95%.

FIG. 2 is panel showing an immunoblot analysis using a monoclonal anti-PDE10A in different bovine reproductive tissues. Protein extracts were separated by SDS-PAGE. Expression of PDE10A protein was detected using a monoclonal PDE10A antibody in rat brain as positive control with immunoreactive bands detected at 77 kDa and 67 kDa. The protein was also detected in bovine testis, epididymis (caput and cauda), seminal vesicle (SV), seminal plasma (SP) and spermatozoa (SPZ). Positive control of human recombinant protein was also loaded (Rec Prot). Representative results from 4 replicates are shown.

FIGS. 3A-H are bar graphs showing family specific 3′-5′ cAMP-PDE activity in bovine testis, caput, cauda and seminal vesicles homogenates. Tissues from bovine Testis (A), Caput (B), Cauda (C) and Seminal Vesicles (D) were subjected to PDE assay after being homogenised in hypotonic buffer. Enzymatic assay of 3′-5′ cAMP-PDE activity of A, B, C and D was measured using 1 μM of cAMP and total PDE activity is represented as the quantity of cAMP (fMol) hydrolyzed per minute per μg of protein (A: 236.51±55.49; B: 215.17±60.83; C, 257.48±64.52; D: 230.79±91.21). Family specific cAMP-PDE activity was also analyzed using different PDE inhibitors to calculate the contribution of different families to the total activity measured. 3-isobutyl-1-methylxanthine (IBMX, 1 mM) inhibits all PDEs using cAMP as substrate except for PDE8; Cilostamide (Cil, 10 μM), Rolipram (Rol, 10 μM) and Papaverine (Papa, 400 nM) specifically inhibit PDE3, PDE4 and PDE10 respectively. Data represented in E, F, G and H are the percentage of total activity sensitive to each inhibitor for bovine Testis, Caput, Cauda and Seminal Vesicles tissues respectively. Data represent the mean±SEM of 3 biological samples of triplicates for each tissue analyzed.

FIGS. 4A and 4B are graphs showing 3′-5′ cAMP-PDE activity in bovine cauda epididymal plasma. 3′-5′ cAMP-PDE activity was measured using different volumes of fresh bovine epididymal plasma (EP). PDE activity is represented as the quantity of cAMP (fMol) hydrolyzed per minute per μg of protein (A). Enzymatic assay of 3′-5′ cAMP-PDE activity of EP was measured using 1 μM of cAMP. As seen in (A) the optimal volume of EP to measure a maximum of 3′-5′ PDE activity is 0.5 μL (A:51.18±13.18). Under 0.5 μL of EP, the activity is measured in the linear range of the assay, i.e. less than 10 000 CPM (B). Data represents the mean±SEM of n=3 biological samples of triplicates.

FIGS. 5A and 5B are bar graphs showing family specific 3′-5′ cAMP-PDE activity in bovine cauda epididymal plasma. To determine which PDE families contribute to the total 3′-5′ cAMP-PDE activity in bovine EP, thawed EP (0.5 μL) samples were incubated in the presence of different PDE inhibitors. PDE activity is represented as the quantity of cAMP (fMol) hydrolyzed per minute per μg of protein. Different PDE inhibitors were used to calculate the contribution of different families to the total activity measured. 3-isobutyl-1-methylxanthine (IBMX, 1 μM) inhibits all PDEs using cAMP as substrate except for PDE8 (which is IBMX-insensitive); Cilostamide (10 μM), Rolipram (10 μM) and Papaverine (400 μM) specifically inhibit PDE3, PDE4 and PDE10 respectively. Data represents the activity sensitive to different PDE inhibitors. Data represented in (B), are the percentage of total activity sensitive to each inhibitor of (A). Data represent the mean±SEM of n=3 biological samples of triplicates.

FIGS. 6A-6C are graphs showing 3′-5′ cAMP-PDE activity in bovine seminal plasma. 3′-5′ cAMP-PDE activity was measured using different volumes of SP from freshly ejaculated bovine sperm. PDE activity is represented as the quantity of cAMP (fMol) hydrolyzed per minute per million of spermatozoa (A) and the quantity of cAMP (fMol) hydrolyzed per minute per μg of protein (B). Enzymatic assay of 3′-5′ cAMP-PDE activity of SP was measured using 1 μM of cAMP. As seen in (A) and (B) the optimal volume of SP to measure a maximum of 3′-5′ PDE activity is 2 μL (A: 603.2±40.8 and B: 11.4±0.9). Under 2 μL of SP, the activity is measured in the linear range of the assay, i.e. less than 20 000 CPM (C). Data represents the mean±SEM of n=18 biological samples of triplicates.

FIGS. 7A and 7B are bar graphs showing 3′-5′ cAMP-PDE activity in fresh and thawed (−80° C.) SP samples. Fresh SP samples that were previously reported and analyzed through enzymatic assay were frozen at −80° C. and thawed once for further characterization. Using 2 μL of SP, PDE activity is represented as the quantity of cAMP (fMol) hydrolyzed per minute per million of spermatozoa (A) and the quantity of cAMP (fMol) hydrolyzed per minute per μg of protein (B). Statistical analysis was performed by Mann Withney t-test (P>0.4). Data represent the mean±SEM of n=10 paired biological samples done in triplicates.

FIGS. 8A-8C are bar graphs showing family specific 3′-5′ cAMP-PDE activity in bovine seminal plasma. To determine which PDE families contribute to the total 3′-5′ cAMP-PDE activity in bovine SP, thawed SP samples were incubated in the presence of different PDE inhibitors. PDE activity is represented as the quantity of cAMP (fMol) hydrolyzed per minute per million of spermatozoa (A) and the quantity of cAMP (fMol) hydrolyzed per minute per μg of protein (B). Different PDE inhibitors were used to calculate the contribution of different families to the total activity measured. 3-isobutyl-1-methylxanthine (IBMX, 1 mM) inhibits all PDEs using cAMP as substrate except for PDE8; Cilostamide (10 μM), Rolipram (10 μM) and Papaverine (400 nM) specifically inhibit PDE3, PDE4 and PDE10 respectively. In both reported values as seen in (A) and (B), data represents the activity sensitive to different PDE inhibitors. Data represented in (C), are the percentage of total activity sensitive to each inhibitor of (A). Data represent the mean±SEM of n=10 biological samples of triplicates.

FIGS. 9A and 9B depict high-performance anion-exchange chromatography of thawed SP and its elution profile, respectively. 1 mL of a single thawed SP sample was loaded onto a HiTrap Q-Sepharose™ column. Prior to the experiment, the column was washed with elution buffer lacking NaCl and the proteins were eluted using elution buffer with a linear NaCl gradient. Fractions (1 mL each) were recovered and were subject to cAMP-PDE assay using cAMP as substrate (1 μM). PDE activity is represented as the quantity of cAMP (pMol) hydrolyzed per minute per fraction. As seen in (A) the highest activity was measured in fraction 18 (20.02 pMol/min/mL) and fractions 12, 15, 18, 21 and 24 were subjected to immunoblot analysis using a monoclonal anti-PDE10A (B) Thawed SP from the same bull was used as a positive control. One representative result of duplicates is presented.

FIGS. 10A and 10B are plot graphs illustrating the linear relationship between PDE activity and fertility index measured on seminal fluid of bull semen. 3′-5′ cAMP-PDE activity was measured in SP from different ejaculated bovine semen. POE activity is represented as the quantity of cAMP (fMol) hydrolyzed per minute per million of spermatozoa (A) and the quantity of cAMP (fMol) hydrolyzed per minute per μg of protein (B). Total PDE activity and papaverine-sensitive activity are plotted according to the fertility index (SOL) obtained from L'Alliance Boviteq Inc. Data are from 28 different bulls.

FIGS. 11A-11D are bar graphs showing regulation of bovine seminal plasma 3′-5′ cAMP-PDE activity following incubation in the presence or absence of spermatozoa. Fresh ejaculates were collected from 4 different bulls on the same day. PDE activity is represented as the quantity of cAMP (fMol) hydrolyzed per minute per million of spermatozoa (A, B) and the quantity of cAMP (fMol) hydrolyzed per minute per μg of protein (C, D). SP was isolated following ejaculation and directly subjected to PDE assay (Ct, A: 395.93±69.07; C, 4.205±0.89). The same assay was also performed on the same SP samples isolated directly after ejaculation but after incubation for three hours at 4° C. (A: 563.05±51.95; C, 6.01±1.19) and 18° C. (A: 526.89±73.11; C, 4.91±0.64). The same assay was also performed on SP samples isolated after three hours of incubation in the presence of spermatozoa at 4° C. (A: 1021.7±111.3; C, 9.15±1.85) and 18° C. (A: 792.79±137.205; C: 7.58±1.05). Family specific cAMP-PDE acitivity was also analysed using different PDE inhibitors to calculate the contribution of different families to the total activity measured. 3-isobutyl-1-methylxanthine (IBMX, 1 mM) inhibits all PDEs using cAMP as substrate except for PDE8; Cilostamide (10 μM), Rolipram (10 μM) and Papaverine (400 nM).

FIGS. 12A-12C are graphs showing 3′-5′ cAMP-PDE activity in bovine spermatozoa. Cyclic AMP-PDE activity was measured in bovine spermatozoa using 1 μM of cAMP. PDE activity is represented either as the count per minute (CPM, A) or as the quantity of cAMP (fMol) hydrolyzed per minute per million of spermatozoa (B). Family specific 3′-5′ cAMP-PDE activity was also analyzed using different PDE inhibitors to calculate the contribution of different families to the total activity measured. 3-isobutyl-1-methylxanthine (IBMX, 1 mM) inhibits all PDEs using cAMP as substrate except for PDE8; Cilostamide (Cil, 10 μM), Rolipram (Rol, 10 μM) and Papaverine (Papa, 400 nM) specifically inhibit PDE3, PDE4 and PDE10 respectively. Data represent the mean±SEM of 29 biological samples done in triplicates. (C) High-performance anion-exchange chromatography of homogenized spermatozoa and its elution profile. 1 mL of a single sample was loaded onto a HiTrap Q-Sepharose column. Prior to the experiment, the column was washed with elution buffer lacking NaCl and the proteins were eluted using elution buffer with a linear NaCl gradient (blue line). Fractions (1 mL each) were recovered and were subject to cAMP-PDE assay. PDE activity (red dots) is represented as the quantity of cAMP (pMol) hydrolyzed per minute per fraction. One representative result of duplicates is presented.

FIGS. 13A-13E are bar graphs showing 3′-5′ cAMP-PDE activity in bovine spermatozoa from freshly ejaculated spermatozoa compared to cryopreserved spermatozoa (paired ejaculates). Cyclic AMP-PDE activity is measured in bovine spermatozoa using 1 μM of cAMP. Total PDE activity in freshly ejaculated spermatozoa is expressed as (A) a mean of all paired biological samples (n=24), (B) in paired biological samples of only different bulls (n=12) and (C) as in (B) and corrected on a positive internal control. Data are plotted as the mean±SEM. Statistical analysis showed a significant difference according to a paired t-test (P<0.05). D-E, Family specific 3′-5′ cAMP-PDE activity in bovine spermatozoa from freshly ejaculated spermatozoa (D) and cryopreserved spermatozoa (E). To determine which PDE families contribute to the total cAMP-PDE activity, bovine spermatozoa were assayed in presence of different PDE inhibitors. The inhibitors were used to calculate the contribution of different families to the total activity measured (total activity minus inhibited activity in presence of PDE inhibitors). 3-Isobutyl-1-methylxanthine (IBMX, 1 mM) to measure all cAMP-PDE except PDE8 family, cilostamide (Cil, 10 μM) for PDE3 family, rolipram (Rol, 10 μM) for PDE4 family and papaverine (Papa, 400 nM) for PDE10 family. Data are the mean±SEM of paired biological samples of different bulls (n=12) and corrected on the positive internal control.

FIGS. 14A-14F depict family specific 3′-5′ cAMP-PDE activity in bovine cauda epididymal spermatozoa. Epididymal spermatozoa from cauda were subjected to PDE assay after being homogenised in hypotonic buffer. Enzymatic assay of 3′-5′ cAMP-PDE activity of epididymal spermatozoa was measured using 1 μM of cAMP and PDE activity is represented as the quantity of cAMP (fMol) hydrolyzed per minute per million of spermatozoa. Family specific cAMP-PDE acitivity was also analyzed using different PDE inhibitors to calculate the contribution of different families to the total activity measured. 3-isobutyl-1-methylxanthine (IBMX, 1 mM) inhibits all PDEs using cAMP as substrate except for PDE8; Cilostamide (Cil, 10 μM), Rolipram (Rol, 10 μM) and Papaverine (Papa, 400 nM) specifically inhibit PDE3, PDE4 and PDE10 respectively. Data represents the activity sensitive to different PDE inhibitors. Data represented in (B) are the percentage of total activity sensitive to each inhibitor. Data represent the mean±SEM of 4 biological samples done in triplicates. C, Immunoblot analysis of PDE10A in cauda, cryopreserved and freshly ejaculated spermatozoa using a monoclonal antibody (Origene). The same concentration of 2 million of spermatozoa was loaded in each lane. One representative experiment of 4 replicates is shown. D, E, F, Immunocytochemistry of PDE10A in bovine spermatozoa. After paraformaldehyde fixation (3.7%) and permeabilisation using triton X-100 (0.2%), the signal was obtained using a polyclonal antibody for PDE10A (Abcam). Two main pattern were obtained, acrosome labelling (D) and both acrosome and post-acrosomal region (E). The pattern was analyzed in both fresh (D-E) and cryopreserved spermatozoa (D-E). The pattern obtained in F in representative of cauda spermatozoa. Data was the mean of 3 replicates.

FIG. 15 is a bar graph showing 3′-5′ cAMP-PDE activity measured in freshly ejaculated bovine spermatozoa after incubation in seminal plasma. Cyclic AMP-PDE activity is measured in bovine spermatozoa using 1 μM of cAMP and PDE activity is represented as the quantity of cAMP (fMol) hydrolyzed per minute per million of spermatozoa. PDE activity was either measured rapidly after ejaculation (0 h), after 3 h of incubation in seminal plasma (SP) either at 4° C. or 18° C., in egg yolk based cryoprotectant (EY) at 18° C. or in liquid semen extender (LS) at 18° C. Statistical analysis was performed by ANOVA (P=0.07). Asterisks indicate significant differences according to Dunnett multiple comparison post-hoc test. Data are the mean±SEM of 4 replicates done in triplicates.

FIGS. 16A-16D are graphs showing linear relationship between 3′-5′ cAMP PDE activity and fertility index measured in fresh and cryopreserved bull spermatozoa. The data are plotted from paired samples (n=12). Total PDE activity (A) and papaverine-sensitive PDE activity (B) in both fresh and cryopreserved spermatozoa is plotted according to the fertility index (Sol). Ratio of total PDE activity (C) and ratio of papaverine-sensitive PDE activity (D) in fresh over cryopreserved is plotted according to the fertility index. Linear regression results in a R square of 0.1451 with a P value of 0.161. Data are the means of triplicates of 12 biological samples.

FIG. 17 is a bar graph showing 3′-5′ cAMP-PDE activity measured in human spermatozoa according to three different fractionation methods. Cyclic AMP-PDE activity is measured in human spermatozoa using 1 μM of cAMP and PDE activity is represented as the quantity of cAMP (fMol) hydrolyzed per minute per million of spermatozoa. PDE activity was either measured according to the G2 method, two-step Percoll gradient (40-80%) or 4 layers Percoll gradient (95%) fractionation. Statistical analysis was performed by ANOVA (P<0.05). Asterisks indicate significant differences according to Dunnett multiple comparison post-hoc test between G2 method and 95%. Data are the mean±SEM of 4 replicates done in triplicates.

FIGS. 18A-18F are bar graphs showing family specific 3′-5′ cAMP-PDE activity measured in human spermatozoa according to three different fractionation methods. Enzymatic assay of 3′-5′ cAMP-PDE activity of ejaculated human spermatozoa was measured using 1 μM of cAMP and PDE activity is represented as the quantity of cAMP (fMol) hydrolyzed per minute per million of spermatozoa. PDE activity was measured in samples isolated by different fractionation methods, G2 methods (A, B), 40%-80% (C, D) and 95% (E, F). Family specific cAMP-PDE acitivity was also analyzed using different PDE inhibitors to calculate the contribution of different families to the total activity measured. 3-isobutyl-1-methylxanthine (IBMX, 1 mM) inhibits all PDEs using cAMP as substrate except for PDE8; Cilostamide (Cil, 10 μM), Rolipram (Rol, 10 μM), Dipyridamole (10 μM) and Papaverine (Papa, 400 nM) specifically inhibit PDE3, PDE4, a group PDE (PDE7, PDE8, PDE10 and PDE11) and PDE10 respectively. Data represents the activity sensitive to different PDE inhibitors (A, C, E). The percentage of PDE activity is represented according to each method (B, D, F). Data are the mean±SEM of a minimum of 3 biological samples done in triplicates.

FIGS. 19A and 19B are bar graphs showing family specific 3′-5′ cAMP-PDE activity in human seminal plasma. To determine which PDE families contribute to the total 3′-5′ cAMP-PDE activity in human seminal plasma, fresh samples were incubated in the presence of different PDE inhibitors. PDE activity is represented as the quantity of cAMP (fMol) hydrolyzed per minute per μg of protein (A). Different PDE inhibitors were used to calculate the contribution of different families to the total activity measured. 3-isobutyl-1-methylxanthine (IBMX, 1 mM) inhibits all PDEs using cAMP as substrate except for PDE8; Cilostamide (10 μM), Rolipram (10 μM), BRL-50481 (3 μM), dipyridamole (10 μM) and Papaverine (400 nM) specifically inhibit PDE3, PDE4, PDE7, a group of PDE (PDE7, PDE8, PDE10 and PDE11) and PDE10, respectively. In reported values as seen in (A), data represents the activity sensitive to different PDE inhibitors. Data represented in (B), are the percentage of total activity sensitive to each inhibitor of (A). Data represent the mean±SEM of 4 biological samples done in triplicates.

FIG. 20 is a panel showing immunoblot analysis in human spermatozoa using different fractionation methods. Protein extracts were separated by SDS-PAGE. Expression of PDE10A, PDE4D and PDE8A protein was detected using a monoclonal anti-PDE10A, a polyclonal anti-PDE4D and a polyclonal anti-PDE8A. Immunoreactive bands were detected at 75 kDa for PDE10A, at 68 kDa for PDE4D, and at 100 kDa and 75 kDa for PDE8A. The letters represent individual donor. The fractionation methods used were either G2 method, 80% or 95%. Tubulin was used as a loading control. The absence of tubulin in liquid seminal (LS) is supporting the absence of contaminating cells.

FIG. 21 is a bar graph showing 3′-5′ cAMP-PDE activity in bovine spermatozoa freshly ejaculated. Cyclic AMP-PDE activity was measured in bovine spermatozoa using 1 μM of cAMP. PDE activity is represented as the quantity of cAMP (fMol) hydrolyzed per minute per million of spermatozoa. Family specific 3′-5′ cAMP-PDE activity was also analyzed using different PDE inhibitors to calculate the contribution of different families to the total activity measured. 3-isobutyl-1-methylxanthine (IBMX, 1 mM) inhibits all PDEs using cAMP as substrate except for PDE8; Cilostamide (Cil, 10 μM), Rolipram (Rol, 10 μM) and Papaverine (Papa, 400 nM) specifically inhibit PDE3, PDE4 and PDE10 respectively. DIpyridamole inhibits PDE7, PDE8, PDE10 and PDE11. Data represent the mean±SEM of 3 biological samples done in triplicates.

### DETAILED DESCRIPTION OF THE INVENTION

### A) Diagnostic Applications

Cyclic nucleotide phosphodiesterases (PDEs) are enzymes which catalyze the hydrolysis of cyclic nucleotides such as cAMP and cGMP. The PDEs thus play critical regulatory roles in a wide variety of signal transduction pathways and PDE inhibitors have long been used for studying sperm-specific processes such as motility, capacitation, agglutination, acrosome reaction and hyperactivation.

Surprisingly, the present inventors have found that sperm phosphodiesterase (PDE) enzymatic activity varies before and after cryopreservation (i.e. a cycle of freezing and thawing) in sperm and that sperm PDE activity correlates with semen quality, semen fertility, and semen resistance to cryopreservation. Thus, measurement of sperm PDE activity before and after freezing can be a useful predictor of sperm function(s).

Accordingly, one aspect of the present invention relates to methods for evaluating a sperm sample by measuring sperm phosphodiesterase (PDE) enzymatic activity in the sample, wherein said PDE activity is indicative of sperm ability to maintain its physiological function(s) during conservation, including but not limited to the following physiological functions: semen quality, semen fertility, sperm viability, sperm motility, sperm capacitation, and semen resistance to cryopreservation. Preferably, the PDE enzymatic activity is measured in isolated spermatozoa.

In one embodiment, the sperm phosphodiesterase PDE enzymatic activity is measured before and after freezing of the sample. Preferably, the PDE enzymatic activity in measured in isolated spermatozoa. In another embodiment, the sperm PDE enzymatic activity which is measured is papaverine sensitive. Preferably, it is the activity of the PDE10 which is measured.

The methods and compositions according to the invention are applicable to mammalian sperm in general. As used herein, the term “sperm” refers to mature ejaculated male gametes, commonly called “sperm” but specifically known as “spermatozoa”, which are able to fertilize the counterpart female gamete. However, the invention is not limited to mature ejaculated sperm and encompasses the use of non-ejaculated sperm and sperm at different stages of their maturation, including but not limited to spermatogoniums (types A and B), primary and secondary spermatocytes and spermatids. Examples of mammalian sperm include, but are not limited to, human sperm, sperm from domestic animals (e.g. bovine sperm, porcine sperm, equine sperm, dog sperm, cat sperm), sperm from laboratory animals (e.g. rat sperm, mouse sperm, rabbit sperm, etc) and sperm from wild animals such as those living in zoos (e.g. lion sperm, tiger sperm, elephant sperm, and the like).

As used herein, the term “sperm sample” refers to any kind of fluid comprising a one or more sperm as defined above. Examples of such fluids include, but are not limited to, biological samples comprising non-ejaculated or comprising ejaculated sperm, sperm ejaculates, culture media comprising sperm, sperm in semen extenders (e.g. extenders for cryopreserving sperm or for preserving sperm in a liquid state), buffers comprising isolated and/or diluted sperm, and the like. Particular examples of sperm samples include, but are not limited to, sperm samples which are prepared during manipulation, preparation, dilution, conservation, freezing, thawing, cell-sorting, and/or sex-sorting of spermatozoa. In preferred embodiments, the sperm sample according to the invention comprises isolated spermatozoa, and more particularly spermatozoa isolated form seminal plasma.

A related aspect of the invention concerns the use of PDE10 as a biomarker of fertility. According to that aspect, enzymatic activity of PDE10 is indicative of the sperm future performance (e.g. survival, motility, fertility, etc), and more particularly such activity is indicative of the sperm resistance to one or more steps of manipulation, dilution, freezing, thawing, cell-sorting, sex-sorting, insemination, and/or fertilization.

In preferred embodiments, the PDE10 enzymatic activity is measured before and after a freezing, then both activities are compared. For instance, and one can calculate a ratio representing the PDE activity before freezing over the PDE activity after freezing. According to the invention, a ratio fresh/frozen is lower in sperm having “low” fertility index (i.e. less fertile spermatozoa). Similarly, a ratio fresh/frozen is higher in sperm having “greater” fertility index (i.e. more fertile spermatozoa) and a ratio fresh/frozen of about 1.5 or higher is predictive of a greater fertility than a ratio of about 1.0 or lower. In some embodiments, the calculated ratio is greater than about 1.25, greater than about 1.5, greater than about 1.75, greater than about 2.0, greater than about 2.5, or greater than about 3.0 for sperm having “greater” fertility index.

It is also conceivable that PDE10 protein levels in sperm could be a biomarker as predictive as PDE10 enzymatic activity. Therefore, some aspects of the present invention encompass measurement of PDE10 protein levels. PDE10 protein levels in sperm could be measured using commonly used methods such as western blot, immunocytochemistry and immunohistochemistry.

Therefore, the invention seeks to benefit from the PDE10 biomarker and to provide methods of screening agents, mixtures, compositions, conditions, processes, etc. to identify those methods or compounds that may be beneficial or that may be harmful to spermatozoa. For instance, assessment of PDE10 activity may be helpful for developing new semen extenders and/or for optimizing current artificial insemination procedures. The invention may also be helpful in optimising the management of semen ejaculates. For instance, by evaluating whether the semen is fit for cryopreservation, one can decide whether a given ejaculate should be frozen or not (i.e. preservation instead as a liquid phase semen).

### B) Therapeutic Applications

Another aspect of the present invention relates to new uses of PDE inhibitors, and more particularly to methods for preserving a mammalian sperm sample (as defined hereinabove), wherein the sample is contacted with an effective amount of a phosphodiesterase (PDE) inhibitor for maintaining sperm physiological functions during conservation. The use of PDE inhibitors according to invention is particularly useful for preserving sperm samples during the many steps of manipulation, preparation, dilution, conservation, freezing, thawing, cell-sorting, and/or sex-sorting of spermatozoa in which such samples may be subjected, especially during in vitro fertilization, artificial insemination and/or cryopreservation.

As used herein “inhibitor” is defined by any molecule/compound/agent that is capable of inhibiting, decreasing, deactivating, antagonizing or eliciting a decrease in activity levels of the target object (e.g. PDE10 enzyme) of the inhibitor. Inhibition of PDE enzymatic activity can be measured by using different methods and assays known in the art. Most common methods, such as those described hereinafter in the Exemplification section, measure degradation of cAMP or cGMP in the sperm. Inhibition of PDE activity can also be assessed by measuring a parameter of sperm function likely affected by reduced PDE activity, including but not limited to motility, survival, fertility, etc.

According to the invention, effective amounts of the PDE inhibitor(s) are being used. As used herein, the term “effective amount” means the amount of a compound that, when contacted with the sperm sample, is sufficient to provide a desired biological effect. The effective amount may vary depending upon many factors, including the identity of the PDE inhibitor, half-life and the biochemical properties of the specific PDE inhibitor employed, the quality or integrity of the semen (e.g. motility, viability), the status of the spermatozoa (e.g. capacitated, acrosome reacted), and the intended used (e.g. cryopreservation, liquid state), the mammalian species of interest, etc. According to the invention, the selected amount is effective in maintaining sperm physiological functions during conservation. In some embodiments, the PDE inhibitor is present at a final concentration of about 50 nM to about 1000 nm, or at a final concentration of about 100 nM to about 800 nM, or at a final concentration of about 400 nM to about 600 nM. For instance, according to particular embodiments with bovine sperm, the use of a specific inhibitor for PDE10 such as papaverine, preferably at a dose of about 400 nM. Such dose is expected to improve the cryopreservation response of spermatozoa, and improve the conservation in a liquid extender or medium by optimizing physiological parameters of spermatozoa (e.g. higher motility, greater viability, more sperm having intact mitochondria, fewer sperm having undergone acrosome reaction or capacitation).

In preferred embodiments, the sperm is contacted rapidly with the PDE inhibitor post ejaculation (e.g. within 60 min or within 30 min, within 5 min post ejaculation, or within 1 min).

### C) PDE Inhibitors

PDE10 is identified as a unique family based on primary amino acid sequence and distinct enzymatic activity. So far, PDE10 has been identified in mouse, human and rats revealing a high degree of homology across species. However, the PDE10 family of polypeptides shows a lower degree of sequence homology as compared to previously identified PDE families and has been shown to be insensitive to certain inhibitors that are known to be specific for other PDE families. PDE10 also is uniquely localized in mammals relative to other PDE families. Messenger RNA for PDE10 is highly expressed in testis and brain (Fujishige, K. et al., Eur J. Biochem. 266:1118-1127, 1999; Soderling, S. et al., Proc. Natl. Acad. Sci. 96:7071-7076, 1999; Loughney, K. et al., Gene 234:109-117, 1999). Moderate expression of PDE10 was also observed in various tissues, including the thyroid and the pituitary glands (Fusjisge et al. J. Biol. Chem. (1999), 274(26), 18438-45; Coskran et al. J. Histochem. Cytochem. (2006), 54(11) 1205-13). So far, eighteen known splice variants of PDE10A have been described (Strick et al., PDE10A: A striatum-enriched, dual-substrate phosphodiesterase. In: Beavo J A, Francis S H, Houslay M D (eds.), Cyclic nucleotide phosphodiesterases in health and disease. Boca Raton: CRC Press Taylor and Francis Group; 2007: 713). Those variants are identical in their C-terminal catalytic domains, but differ in size of the N-terminal portion of the molecule. For convenience, and unless otherwise stated, reference herein to “PDE10” will include reference to all forms and variants of PDE10, including those known as PDE10A, PDE10A1 to PDE10A18, PDE10A-b, (see for instance US 2006/166316) and/or others yet unknown or uncharacterized forms and variants, the PDE10 originating from whichever mammal, and more particularly from human sperm, bovine sperm, porcine sperm, equine sperm, dog sperm, cat sperm, rat sperm, or mouse sperm.

In preferred embodiments, the PDE inhibitor according to the invention selectively inhibits PDE10, i.e. it is specific. One example of a preferred selective PDE10 inhibitor is papaverine. In other embodiments, less selective inhibitors such as dipyridamole, an inhibitor of PDE5, PDE6, PDE7, PDE8, PDE10 and PDE11, are used. Some embodiments of the invention encompass inhibition of any of the signalling pathways of which the PDE10 is a member.

There are many PDE10 known in the art that may be useful according to the invention. Selected examples of known selective and non-selective PDE inhibitors used for studying sperms include pentoxifylline, rolipram, IBMX, and papaverine. Dipyridamole and ibudilast are additional selective inhibitors of PDE10. Selective PDE10 inhibitors and methods for screening such inhibitors are described in Chappie et al. (J. Med. Chem. (2007), 50:182-185), Kehler et al. (Expert Opinion Ther. Patents. (2007), 17(2):147-158), US patent publications US 2007/287707; US 2007/265258; US 2007/265270; US 2007/265256; US 2007/155779; US 2006/154931; US 2006/019975; US 2003/008806, and in PCT patent publications WO 2007/129183; WO 2007/103260; WO 2007/096743; WO 2007/022280; WO 2006/089815; WO 2006/094933; WO 2006/0949942; WO 2006/028957; WO 2005/120514; WO 2005/002579, WO 2005/003129; WO 2005/120474 and WO 2004/050091 which are all incorporated herein by reference. Additional known PDE10 inhibitors include, but are not limited to, pyrazoloquinolines (see McElroy et al. Bioorg Med Chem Lett. 2012 Feb. 1; 22(3):1335-9. Epub 2011 Dec. 21., incorporated herein by reference), MP10 (see Langen B, et al. Psychopharmacology (Berl). 2011 Nov. 16. incorporated herein by reference), TP-10 also known as 2-{4-[-pyridin-4-yl-1-(2,2,2-trifluoro-ethyl)-1H-pyrazol-3-yl]-phenoxymethyl}-quinoline succinic acid (see Strick C. A et al. (2007). “PDE10A: a striatum-enriched, dual-substrate phosphodiesterase,” in Cyclic Nucleotide Phosphodiesterases Health Disease, eds Beavo J. A., Francis S. H., Houslay M. D., editors. (Boca Raton, Fla.: CRC Press), 237-254; and also Schmidt C. J. et al. (2008). J. Pharmacol. Exp. Ther. 325, 681-690. doi: 10.1124/jpet. 107.132910, both manuscripts being incorporated herein by reference), and 1-(4-(2-(2-fluoroethoxy)ethoxy)-3-methoxybenzyl)-6,7-dimethoxyisoquinoline (see Zhang Z et al., Eur J Med Chem. 2011 September; 46(9):3986-95. Epub 2011 Jun. 12, incorporated herein by reference). Accordingly, in some embodiments, the PDE inhibitor is selected from the group consisting of dipyridamole, pyrazoloquinolines, MP10, 2-{4-[-pyridin-4-yl-1-(2,2,2-trifluoro-ethyl)-1H-pyrazol-3-yl]-phenoxymethyl}-quinoline succinic acid, and 1-(4-(2-(2-fluoroethoxy)ethoxy)-3-methoxybenzyl)-6,7-dimethoxyisoquinoline.

The use of PDE inhibitors according to invention is particularly useful for preserving sperm samples during the many steps of manipulation, preparation, dilution, freezing, thawing, cell-sorting, and/or sex-sorting of spermatozoa in which such samples may be subjected, especially during in vitro fertilization, artificial insemination and/or cryopreservation. According to another aspect of the invention, the PDE10 inhibitor is used as an active agent for delaying sperm capacitation and/or sperm acrosome reaction.

The present invention further encompasses the use of compounds capable of modifying the composition of the sperm membrane phospholipids, compounds capable of modifying the membrane cholesterol to phospholipid ratio, and/or compounds capable of improving sperm cryosurvival, those compounds being used alone or in combination with a PDE10. Examples of such compounds include, but are not limited to, trehalose, methyl-β cholesterol-loaded-cyclodextrin, glycerol, and raffinose.

### D) Semen Extenders

Another aspect of the present invention concerns a composition (e.g. a semen extender) for preserving a mammalian sperm sample and/or for maintaining sperm physiological functions during conservation, the composition comprising an effective amount of a phosphodiesterase (PDE) inhibitor. A related aspect concerns the use of phosphodiesterase inhibitors for the manufacture of a semen extender. As used herein, the term “semen extender” refers to any type of solution for preserving sperm including, but not limited to, culture media, diluents, buffers, and the like. The semen extender may be formulated for cryopreserving sperm or for preserving sperm in a liquid state for a short or extended period.

In one embodiment, the semen extender is for semen preserved in a liquid state (i.e. liquid phase semen). In one embodiment, the semen extender is for use during any of the steps of in vitro fertilization, including but not limited to humans, in which the sperm are manipulated. In another embodiment, the semen extender is for use during the cryopreservation of the sperm. The amount of PDE inhibitor in composition of the present invention is an effective amount. An effective amount of PDE inhibitor is that amount of inhibitor necessary so that inhibition of PDE activity can be assessed in a sperm sample. PDE activity can be assessed directly using commonly used methods and assays as those described in the Exemplification section, or indirectly by measuring a parameter of sperm function likely affected by such treatment (e.g. motility, survival, fertility, etc.). For example, conventional assays are available to measure (e.g. quantitative) intracellular levels of cAMP or cGMP. The exact amount of PDE inhibitor to be used will vary according to factors such as the number of spermatozoa in the sample, the final sperm concentration, sperm species, the type of manipulation, as well as the other ingredients in the composition. For instance, it has been reported that PDE10A is sensitive to papaverine with an estimated Kᵢ of 30 nM (Strick et al., supra). Typically, the amount of PDE inhibitor could vary from about 0.1 nM to about 1 μM, or from about 100 nM to about 800 nM, of from about 400 nM to about 600 nM. In a preferred embodiment, the PDE inhibitor is papaverine and it is present in the composition in an amount varying from about 10 nM to about 50 μM, preferably from about 100 nM to about 800 nM, and more preferably from about 400 nM to about 600 nM. Further active agents can be added to the composition of the invention. For instance, the composition of the invention may also comprise additional selective inhibitors (see hereinbefore) and/or compounds such as trehalose, methyl-β cholesterol-loaded cyclodextrin, glycerol, and raffinose.

In addition to the active agents (e.g. PDE10 inhibitors and/or additional inhibitors), the compositions of the invention may also contain metal chelators (proteinic or not), metal scavengers (proteinic or not), coating agents preserving agents, solubilizing agents, stabilizing agents, wetting agents, emulsifiers, sweeteners, colorants, odorants, salts, buffers, coating agents and/or antioxidants. For preparing such the composition of the invention, methods well known in the art may be used. For instance, semen extenders typically comprise egg yolk, glycerol, tris, acid citric monohydrate, glucose and antibiotics, they have an osmotic pressure varying from 280 to 300 mOsmol, and a pH varying from 6.7 to 7.4.

### E) Kits

Given the correlation which exists between sperm phosphodiesterase (PDE) activity and semen quality, a related aspect of the invention relates to kits for assessing the impact of manipulation, preparation, dilution, freezing, thawing, cell-sorting, and/or sex-sorting etc., on at least one fertility-related parameter such as semen quality, sperm survival, semen fertility, semen resistance to cryopreservation, fertilization rates, etc.

According to a further aspect, the invention relates to a commercial package or kit for measuring PDE10 enzymatic activity in sperm. A kit of the invention can comprise one or more of the following elements: a buffer for homogenization of the sperm sample(s), PDE10 and/or PDE11 purified proteins to be used as controls, incubation buffer(s), PDE substrate and assay buffer(s), inhibitor buffer(s) and inhibitors (e.g. IBMX) standards (e.g. 5′-AMP, 5′-GMP), 5′-nucleosidase (e.g. from Croatalus atrox venom), detection materials (e.g. antibodies, fluorescein-labelled derivatives of cAMP, luminogenic substrates, detection solutions, scintillation counting fluid, etc), laboratory supplies (e.g. desalting column, reaction tubes or microplates (e.g. 96- or 384-well plates), a user manual or instructions, etc.

The kits of the invention may be particularly useful for applications in animals and humans according to the evaluation methods described hereinbefore. More particularly, the kits disclosed may be helpful for laboratory and diagnostic purposes in humans during artificial insemination procedures.

The kits and methods of the invention may be used in combination with previously described PDE assay kits (e.g. FabGenix PDE4 enzymatic kit, Promega PDE-Glo™ phosphodiesterase assay, Biomol® International cyclic nucleotide phosphodiesterase assay kit AK-800™, Molecular Devices HEFP™ phosphodiesterase assay kit). The invention also encompasses kits for preserving mammalian spermatozoa comprising appropriate amounts of PDE inhibitor(s) in culture media, buffers, diluent, and the like.

### F) Contraceptive Applications

Although the disclosure of the present invention focuses primarily on improving sperm fertility, those skilled in the art will also understand that the principles of the present invention can be readily applied for opposite purposes. For instance, it is conceivable according to the invention to use compounds capable of stimulating or increasing PDE activity as spermicidal agents. Such spermicidal agents could be incorporated in gels, creams or in condoms. Therefore, the invention also encompasses those concepts.

Those skilled in the art will recognize, or be able to ascertain using no more than routine experimentation, numerous equivalents to the specific procedures, embodiments, claims, and examples described herein. Such equivalents are considered to be within the scope of this invention and covered by the claims appended hereto. The invention is further illustrated by the following examples, which should not be construed as further limiting.

### EXAMPLES

The examples set forth herein below provide exemplary methods for evaluating the physiological status of sperm before and after cryopreservation. Also provided are exemplary protocols, molecular tools, probes, primers and techniques.

### Example 1

### Expression of PDE10A in Bovine Tissues and Characterization of PDE10 Enzymatic Activity in Bovine and Human Spermatozoa

### Materials and Methods

Unless mentioned, all chemicals were purchased from Sigma-Aldrich Canada Ltd (Oakville, Ont).

### Bovine Epididymal Plasma and Seminal Plasma Isolation

Epididymis from sexually mature bulls was obtained from a local slaughterhouse. Immediately after slaughtering, testicles were kept on ice and brought to the laboratory within 3 hours. Epididymis was dissected and cleared from connective tissues. As already described (Frenette, G. et al., (2002). Biol Reprod 67, 308-313), fluid from the cauda epididymis was collected by retrograde flushing. Epididymal plasma was isolated according to the same centrifugations steps than the seminal plasma as described below.

Fresh ejaculated bovine sperm, at known concentrations, was transported for three hours at 18° C. Upon arrival specimens were centrifuged at 400×g during 10 minutes at room temperature to gently pull down spermatozoa. Supernatant was transferred and centrifuged again at 6 000×g at 4° C. during 20 minutes to pull down any remaining spermatozoa. Supernatant was then transferred and centrifuged at 6 000×g at 4° C. during 20 minutes to remove any remaining debris. Seminal plasma (SP) samples were kept on ice until used for PDE assay and stored at −80° C. for further characterization.

### Isolation and Preparation of Epididymal Cauda Spermatozoa

Epididymis from sexually mature bulls was obtained from a local slaughterhouse. Immediately after slaughtering, testicles were kept on ice and brought to the laboratory within 3 hours. Epididymis was dissected and cleared from connective tissues. As already described (Frenette, G. et al., (2002). supra), fluid from the cauda epididymidis was collected by retrograde flushing. Spermatozoa concentration from the cauda region was measured by the hemacytometer method and was corrected to obtain a concentration of 1×10⁶ spermatozoa/10 μL homogenized in hypotonic buffer (20 mM Tris-HCl, pH 7.4; 1 mM EDTA; 0.2 mM EGTA; 50 mM sodium fluoride; 50 mM benzamidine; 10 mM sodium pyrophosphate; 4 μg/ml aprotinin; 0.7 μg/ml pepstatin; 10 μg/ml soybean trypsin inhibitor; 0.5 μg/ml leupeptin, 2 mM PMSF; and 0.5% (v/v) Triton™ X-100) using the Dounce homogenizer (30 strokes on ice). Crude samples were subjected to PDE assay.

### Spermatozoa

The semen was obtained from different Holstein bulls. The fresh ejaculated semen was kept in an insulated thermos flask, at 18° C., during transportation to the laboratory and processed within 3 hours after collection. The cryopreserved semen was obtained at the Centre d'Insémination Artificielle du Québec (CIAQ) in St-Hyacinthe. The cryopreserved straws of semen were prepared according to CIAQ procedures.

### Sample Preparation for Use in the “G2 Method”

Thawing was achieved by immersing the straws in 37° C. water bath for 1 minute. After cutting one end of the straw the semen was collected in a 15 ml falcon while cutting the other end. From this point fresh and cryopreserved semen were processed similarly. Approximately 50 μL of the semen was added to 5 mL of Talp-BSA medium in a 15 mL falcon tube to wash the spermatozoa. After centrifugation (280×g, 10 min, room temperature), the remaining pellet of approximately 100 μL was resuspended in Talp-BSA medium (500 μl for the fresh and 200 μl for two straws) to reach a concentration of 1×10⁶ cells/10 μL. The samples were then vortex-agitated for 30 sec and kept on the ice until ready to run the assay. The concentration of sperm cells was measured according to hemacytometer method.

### Preparation of Freshly Ejaculated Human Spermatozoa

Ejaculates were obtained by masturbation from healthy donors after a minimum of 2 days of sexual abstinence. After ejaculation, semen was kept at room temperature to allow liquefaction. To prepare human ejaculate, three different fractionation methods were used: “G2 method”, “40-80% fractionation” or “95% fractionation”.

For “G2 method”, according to the ejaculate volume, approximately 1 ml of semen was added to 5 ml of isotonic solution: HEPES-buffered saline (HBS: 25 mM Hepes, 130 mM NaCl, 4 mM KCl, 0.5 mM MgCl₂, 14 mM fructose, pH 7.6) in a 15 mL falcon tube to wash the spermatozoa from the seminal plasma. After centrifugation (280×g, 10 min, room temperature), the medium are aspirated with a remaining pellet of approximately 100 uL. The sperm cells were then resuspended in HBS to reach a concentration of 0.5×10⁶ cells/10 μl. Then, the samples were vortex-agitated for 30 sec and kept on the ice until ready to run the assay. The concentration of sperm cells was measured according to hemacytometer method.

In the “40-80% fractionation” method, a simplified two-step (40-80%) Percoll gradient (GE healthcare, Baie d'Urfé, Qc, Canada) was used to separate human ejaculates into two populations of spermatozoa. Percoll™ was centrifuged (1000×g, 30 min, room temperature) to separate populations and wash sperm from the seminal plasma. Sperm cells were collected at the bottom of the 80% Percoll fraction and process as in the G2 method.

In the “95% fractionation” method, the semen was layered on top of a Percoll gradient composed of 2 ml fractions each of 20%, 40%, 65% and 100 μl of 95% Percoll in isotonic solution as described above. Sperm cells at the 65-95% interface and in the 95% pellet were collected. The following steps were as in the 40-80% method.

### Human Seminal Plasma Isolation

A certain amount of complete ejaculate was kept to isolate spermatozoa from seminal plasma. This portion was centrifuged at 400×g during 10 minutes at room temperature to gently pull down spermatozoa. Supernatant was transferred and centrifuged again at 6 000×g at 4° C. during 20 minutes to pull down any remaining spermatozoa. Supernatant was then transferred and centrifuged at 6 000×g at 4° C. during 20 minutes to remove any remaining debris. SP samples were kept on ice until used for PDE assay and stored at −80° C. for further characterization.

### RNA Isolation, Reverse Transcription and Polymerase Chain Reaction

Total RNA was isolated from bovine testis, seminal vesicles, caput and cauda tissues obtained from a local slaughterhouse. Upon arrival, small pieces of tissue were dissected on ice, immediately frozen in liquid nitrogen and kept at −80° C. until processed for RNA extractions. Total RNA from tissues was obtained by the Tryzol extraction method and purified using the PicoPure™ RNA Isolation Kit (Molecular Device, catalog #KIT0204). RNA concentration for each tissue was measured using NanoDrop ND-1000™ (NanoDrop Technologies). One μg of RNA for each tissue was then reverse transcribed into cDNA using the qScript FIex™ cDNA Synthesis Kit's random primer (Quanta Biosciences) according to the manufacturer's manual. PCR reactions were carried out using the following cycles: denaturation at 95° C. for 1 minute, annealing at 60° C. for 1 minute, extension at 72° C. for 1 minute for a total of 40 cycles with a final extension of 10 minutes at 72° C. FastStart™ Taq DNA polymerase from Roche was used. The PCR primers were designed according to the predicted Bos taurus phosphodiesterase 10A (PDE10A, Accession number: XM_{—}582454). Negative control was done without cDNA template. The PCR products were separated by gel electrophoresis in 1% agarose, stained with ethidium bromide and photographed with and ultraviolet camera.

### Tissues Preparation for PDE Assay

Testis, Caput, Cauda and Seminal Vesicle tissues were thawed in homogenisation buffer: 1 mL of hypotonic buffer (20 mM Tris-HCl, pH 7.4; 1 mM EDTA; 0.2 mM EGTA; 50 mM sodium fluoride; 50 mM benzamidine; 10 mM sodium pyrophosphate; 4 μg/ml aprotinin; 0.7 μg/ml pepstatin; 10 μg/ml soybean trypsin inhibitor; 0.5 μg/ml leupeptin, 2 mM PMSF; and 0.5% (v/v) Triton™ X-100) using the Dounce homogenizer (30 strokes on ice). After thoroughly vortex-agitating for about one minute, samples were centrifuged at 10 000×g for 4 minutes at room temperature. Supernatant fluid was collected and diluted in different volumes of hypotonic buffer depending on the tissue. Testis, caput and cauda homogenates were diluted in 1 mL of hypotonic buffer while seminal vesicle homogenates were diluted in 2 mL of hypotonic buffer. Homogenates were kept on ice until subjected to PDE assay.

### PDE Activity Measurement

The cAMP enzymatic assay (PDE assay) was conducted as previously described (Sasseville, M. et al., (2006). BMC developmental biology 6, 47). Samples were directly subjected to PDE assay. 3′-5′ cAMP-PDE activity was assessed at 34° C. in 200 μL final volume using 1 μM cAMP as substrate (Thompson, W. J. et al., (1979). Adv Cyclic Nucleotide Res 10, 69-92) with minor modifications. The solution included: 40 mM TrisHCl pH 8.0, 10 mM MgCl₂, 5 mM 2-mercaptoethanol, 0.75 mg/mL BSA (fraction V), 1 μM cold cAMP and 15 nM (H³)cAMP (Perkin Elmer) (1×10⁵ cpm/tube; 30 Ci/mmol). Knowing the exact concentrations of spermatozoa in each SP sample, total 3′-5′ cAMP-PDE activity was reported as the quantity of cAMP (fMol) hydrolysed per minute per the number of spermatozoa (fMol/min/10⁶ spz). Measurements were also performed in the presence of PDE inhibitors: IBMX (1 mM, non-specific), cilostamide (10 μM, PDE3-specific), rolipram (10 μM, PDE4-specific), papaverine (400 nM, PDE10-specific). Cilostamide and Rolipram were purchased from Biomol. To determine which PDE family contributes to total 3′-5′ cAMP-PDE activity, the activity measured in the presence of the respective inhibitors was subtracted from the total. Each PDE assay was performed in triplicate for each experiment and each experiment was repeated at least three times.

### PDE Inhibitors

As described (Sasseville, M. et al., (2009). Biol Reprod 81, 415-425), 3-Isolbutyl-methylxanthine (IBMX) is a broad-spectrum PDE inhibitor that inhibits 3′-5′ cAMP-PDE activity from different PDE families (PDE1, PDE2, PDE3, PDE4, PDE7, PDE10 and PDE11) except for PDE8. Therefore, IBMX-sensitive 3′-5′ cAMP-PDE activity represents the combined inhibited 3′-5′ cAMP-PDE activities of PDE1, PDE2, PDE3, PDE4, PDE7, PDE10 and PDE11 families, whereas IBMX-insentitive cAMP-PDE activity corresponds to PDE8 activity. IBMX-sensitive PDE5 and PDE6 and IBMX-insensitive PDE9 activities are absent from these groups because cGMP is the specific substrate and then are not detected in the cAMP-PDE assay. Cilostamide, Rolipram and Papaverine were used respectively as specific PDE3, PDE4 and PDE10 inhibitors, as described previously (Dostaler-Touchette, V. et al., (2009). J Dairy Sci 92, 3757-3765; Sasseville, M., et al. (2007). Endocrinology 148, 1858-1867; Siuciak, J. A. et al., (2006). Neuropharmacology 51, 386-396).

### High Performance Anion-Exchange Chromatography

A HiTrap-Q Sepharose™ column (GE healthcare) was equilibrated with 10 mL of elution buffer (20 mM Tris-HCl pH 7.4, 1 mM dithiothreitol, 1 mM calcium chloride, 2 μM leupeptin and 5 mM benzamidine) and a preparation of homogenized spermatozoa in hypotonic buffer (described above) was directly loaded. The proteins were eluted from the column by running a linear NaCl gradient (0-400 mM first, 40 mL and 400-1000 mM last, 10 mL) in elution buffer (Fujishige, K. et al., (1999). Eur J Biochem 266, 1118-1127). Fractions (1 mL each) were collected at 4° C. and 20 μL of each fraction was subjected to PDE assay.

### Western Blots

Protein samples were loaded in sample buffer [60 mM Tris-HCl (pH 6.8), 10.5% (vol/vol) glycerol, 2% (wt/vol) SDS, 0.005% (wt/vol) bromophenolblue, and 5% (vol/vol) 2-mercaptoethanol] onto a 10% polyacrylamide gel for electrophoresis. Proteins were transferred to an Immobilon-P™ transfer PVDF membrane (Millipore, Temecule Calif., USA) using a Mini Protean 3 Cell™ apparatus (Bio-Rad Laboratories, Mississauga, ON, Canada). Membranes were then blocked with TBS containing 0.1% (v/v) Tween-20™ and 5% (w/v) none-fat dry milk for one hour. Membranes were blotted with the primary antibody in TBS containing 0.1% (v/v) Tween-20 and 5% (w/v) nonfat dry milk at room temperature for 1 h. The primary antibodies were monoclonal mouse antibody PDE10A (diluted 1:500; Origene), polyclonal PDE4D (diluted 1:200, Santa Cruz), polyclonal PDE8A (Diluted 1:500, Santa Cruz) after which the membranes were exposed to goat anti-mouse peroxydase-conjugated secondary antibody (diluted 1:20 000; Millipore) or rabbit anti-goat (1:30 000 from Jackson Immuno Research) for PDE4D or a goat anti-rabbit anti-rabbit (1:40 000 from Jackson Immuno Research) at room temperature. Binding was detected using the ECL Plus kit (GE Healthcare) and exposed on autoradiograph films (GE Healthcare) or read on Fx7™ camera (Vilber).

### Statistical Analysis

PDE activities were expressed as the means±SEM. PDE activities between fresh and cryopreserved were compared with a paired t-test using GraphPad Prism™ ver. 5.0 for Windows (GraphPad Software, San Diego, Calif.). The effect of incubation on PDE activities were analyzed by one-way ANOVA.

### Results

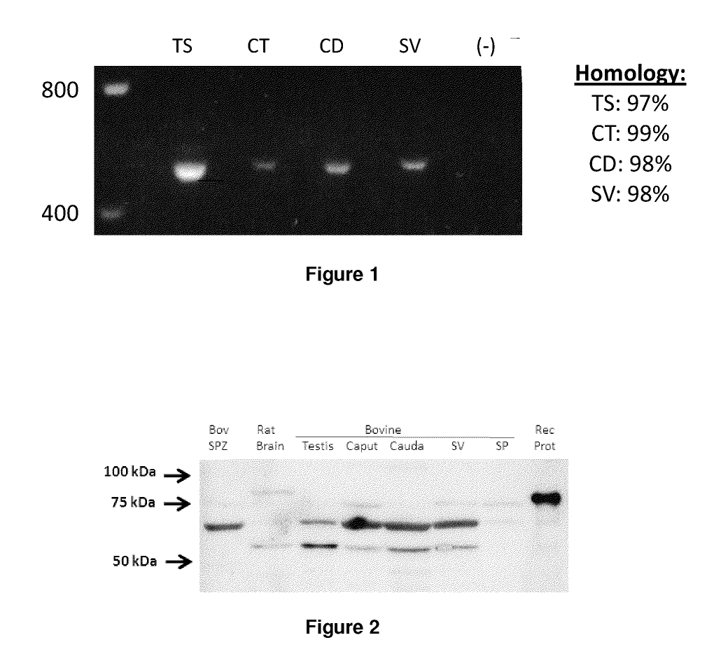

FIG. 1: This figure shows the expression of PDE10A (phosphodiesterase 10A) transcript in different bovine male reproductive tissues. The homology is higher than 95% demonstrating that PDE10A is expressed in bovine testis, epididymis (caput and cauda) and seminal vesicle.

FIG. 2: Immunoblots of PDE10A in different bovine male reproductive tissues. A recombinant PDE10A and positive controls were used to support the expression profile. Two immunological bands were found at 67 kDa and 77 kDa supporting the presence of PDE10A protein in bovine testis, epididymis (caput and cauda), seminal vesicle and seminal plasma.

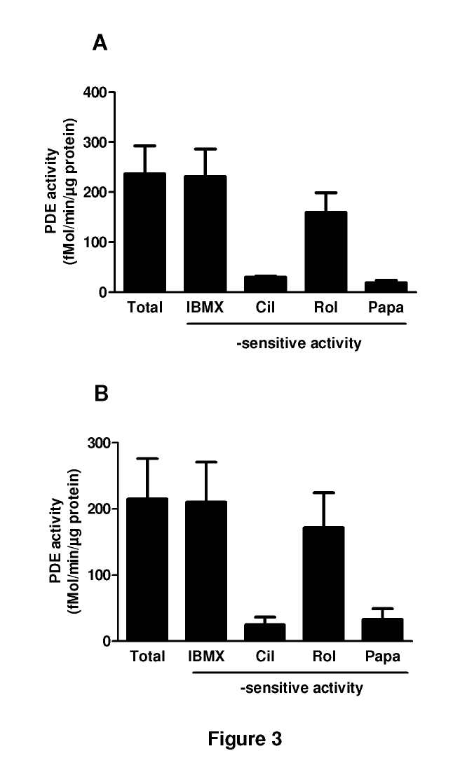

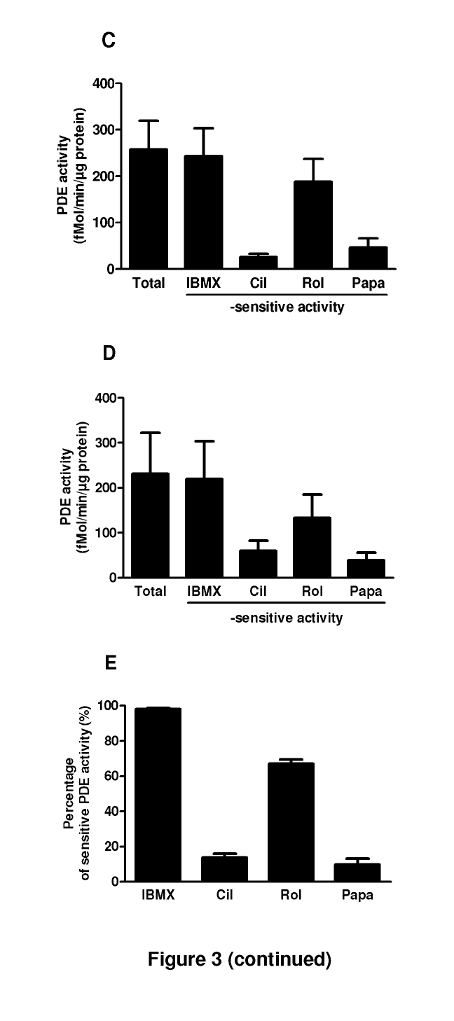

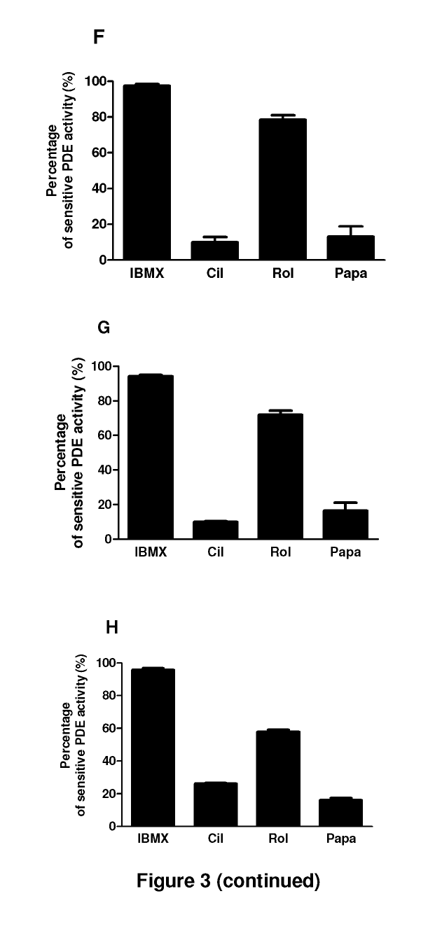

FIG. 3: PDE activity was measured in different bovine male reproductive tissues. Papaverine-sensitive PDE activity (PDE10) was measured with low percentage in all of these tissues. Rolipram-sensitive PDE activity was highly functional supporting the functional activity of PDE4 family but a relatively low contribution of PDE10 in cAMP-PDE activity measured in these tissues.

Taken together, FIGS. 1-3 demonstrate that the transcript of PDE10A is present, the protein is also present but this family of PDE (papaverine-sensitive) contributes with a low percentage of the 3′-5′ cAMP-PDE activity in these tissues.

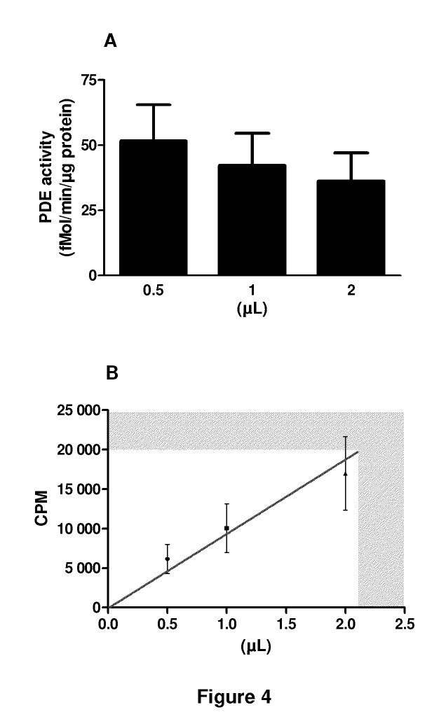

FIG. 4: Technical consideration to determine the volume of sample to be used in bovine epididymal fluid in the assay.

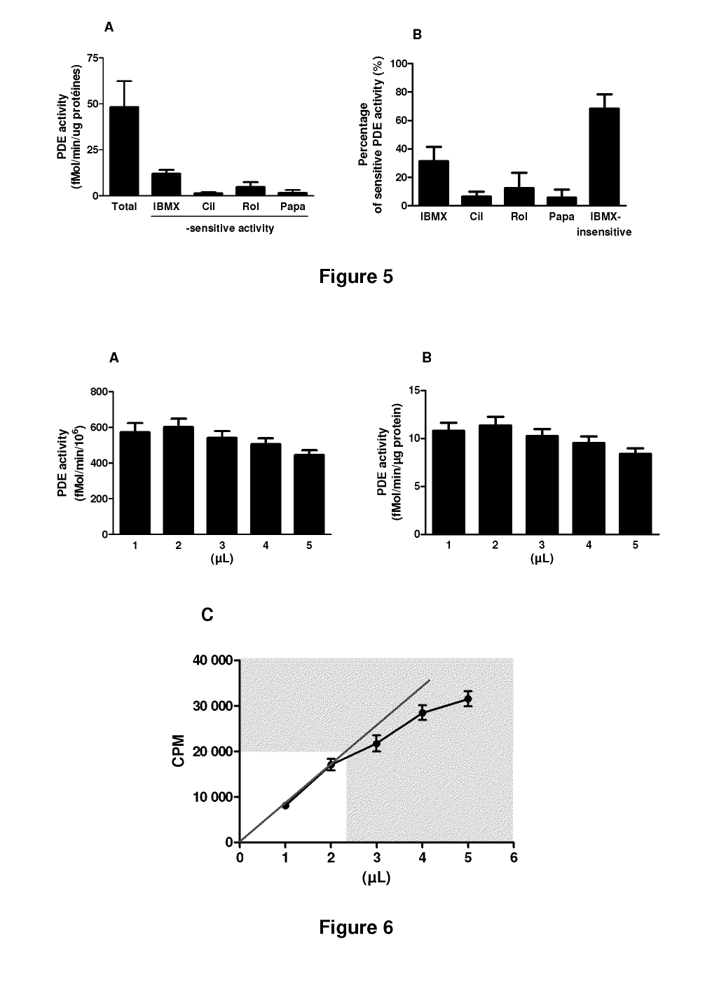

FIG. 5: PDE activity was measured in bovine epididymal fluid. IBMX-insensitive PDE activity supports the functional presence of PDE8 family in epididymal fluid. However, a low papaverine-sensitive PDE activity suggests the absence of significant contribution of PDE10 activity in epididymal fluid which further supports the tissue-specific functional expression.

FIG. 6: Technical consideration to determine the volume of sample to be used in bovine seminal fluid.

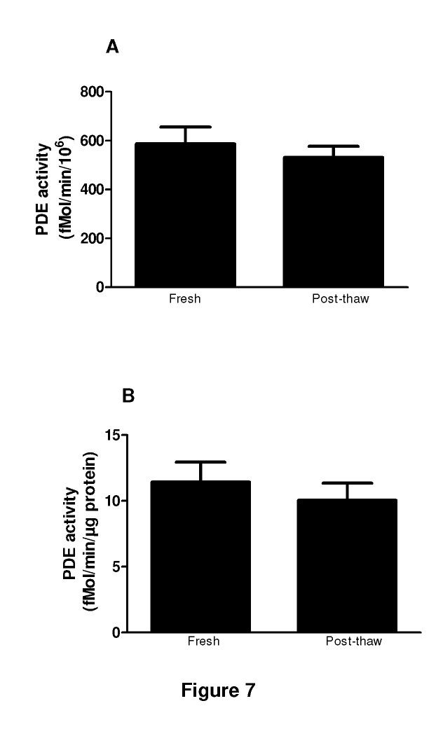

FIG. 7: Comparison of PDE activity between paired sample of fresh and frozen seminal plasma samples. The data suggest no significant difference and supports the ability to further study PDE activity in frozen seminal plasma.

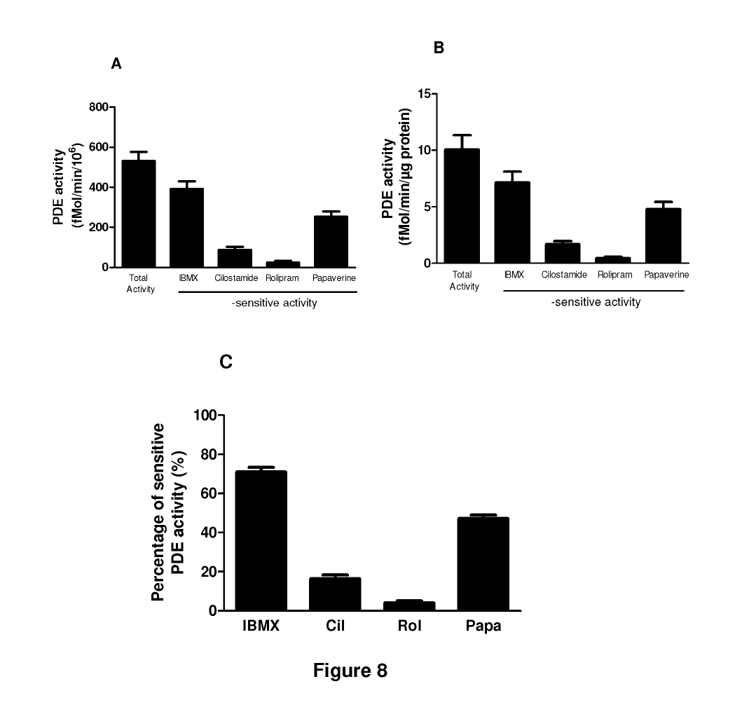

FIG. 8: Family specific PDE activity in bovine seminal plasma. The data show that more than 50% of the PDE activity measured in bovine seminal plasma is papaverine-sensitive, supporting the functional presence of PDE10 in seminal plasma.

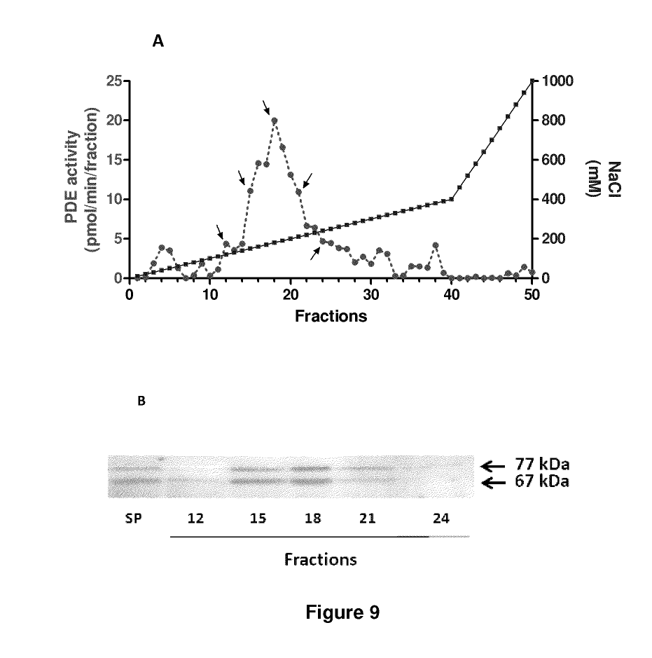

FIG. 9: High performance anion-exchange chromatography of seminal plasma to separate PDE activity according to a salt gradient. The data shows a major peak of PDE activity measured in fraction 18 where a strong immunological band of 77 kDa was revealed which support the presence of a functional PDE10A at 77 kDa in seminal plasma. This is the first demonstration of the functional presence of PDE10A in a biological fluid, confirming that PDE10A may be used as a marker of fertility to discriminate bulls having a low fertility index. Taken together, FIGS. 8 and 9 shows that PDE10A is present and functional in bovine seminal plasma.

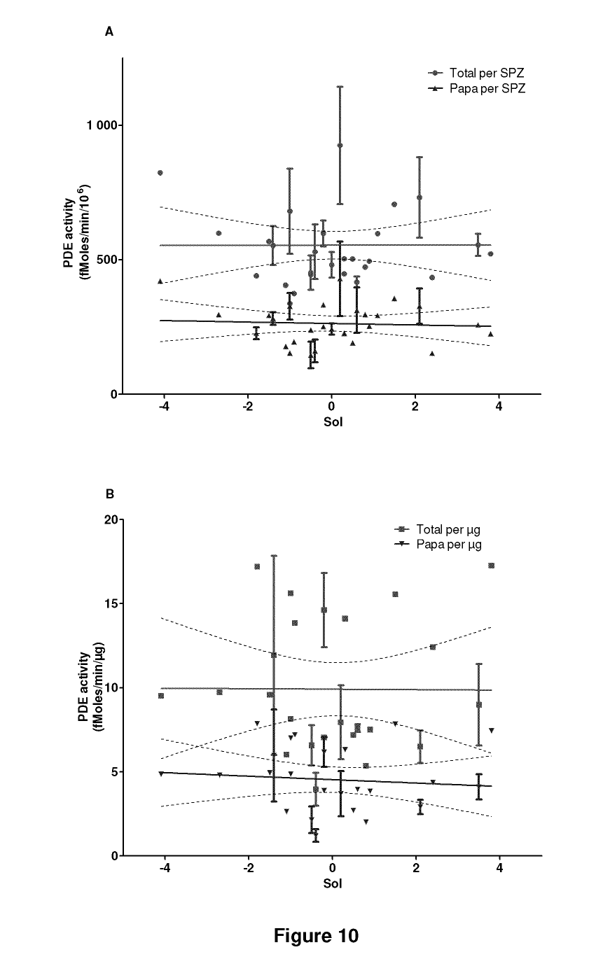

FIG. 10: Linear relationship between PDE activity (total PDE activity and papaverine-sensitive PDE activity) and fertility index of the bulls plotted as SOL. PDE activity measured in seminal plasma is not changing according to the fertility index (SOL).

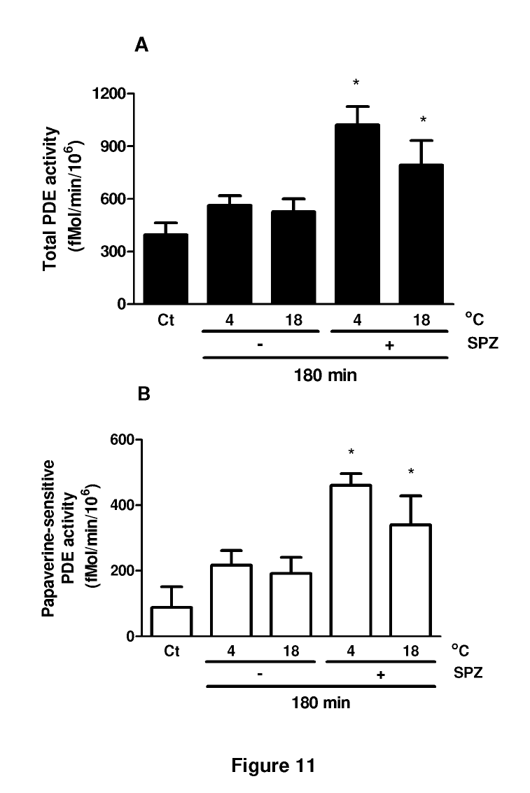

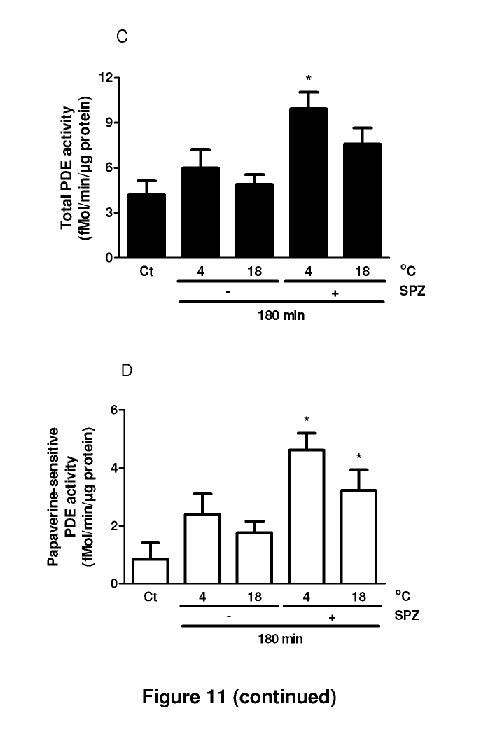

FIG. 11: Regulation of PDE activity in bovine seminal plasma after exposure to spermatozoa. The data shows a significant increase of PDE activity (total and papaverine-sensitive) after incubation in presence of spermatozoa supporting an important regulation of PDE10 activity after ejaculation in seminal plasma while incubated in presence of spermatozoa.

Taken together, FIGS. 10 and 11 suggest that, even in the absence of an observed a correlation between the fertility index and the PDE activity measured in seminal plasma, one can observe a clear impact of the presence of spermatozoa on PDE activity in seminal plasma. These results suggest that a regulation of PDE activity in seminal plasma occurs and that assessment of the PDE activity may be used to discriminate bulls with different fertility index.

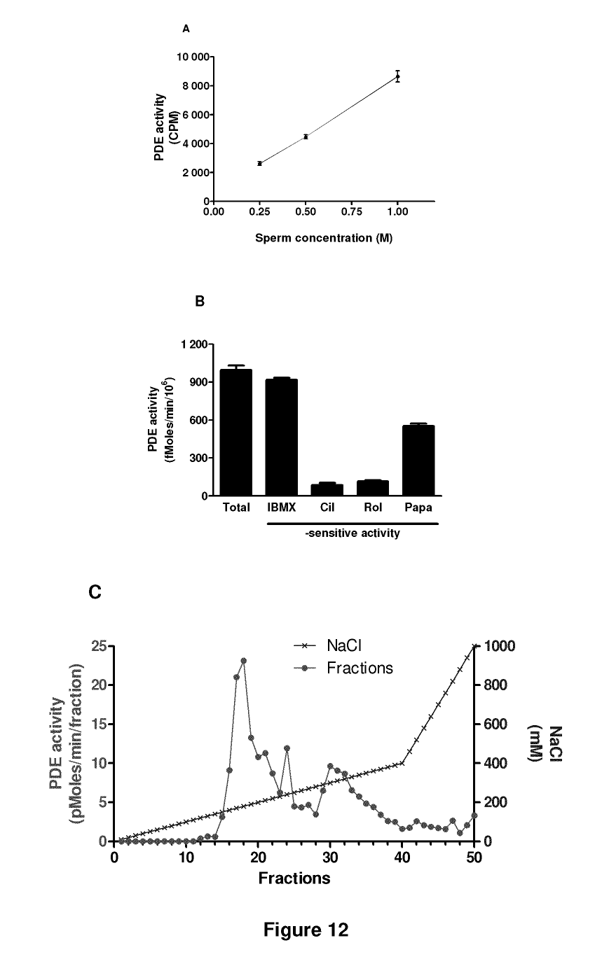

FIG. 12: PDE activity was measured in bovine spermatozoa. The data shows papaverine-sensitive PDE activity. High performance anionic-exchange chromatography was performed and the results support the functional presence of PDE10 in bovine spermatozoa. The data of FIG. 12B are also presented in Table 1 and supports the important contribution of PDE10 measured in bovine spermatozoa, as illustrated by 55% of papaverine-sensitive PDE activity.

TABLE 1
Summary of the contribution of different PDE inhibitors to the total PDE
activity measured in bovine spermatozoa.

| PDE inhibitors | PDE families | Percentage |
|---|---|---|
| IBMX-sensitive | 1, 2, 3, 4, 7, 10, 11 | 92% |
| IBMX-insensitive | 8 | 8% |
| Cilostamide-sensitive | 3 | 9% |
| Rolipram-sensitive | 4 | 11% |
| Papaverine-sensitive | 10 | 55% |

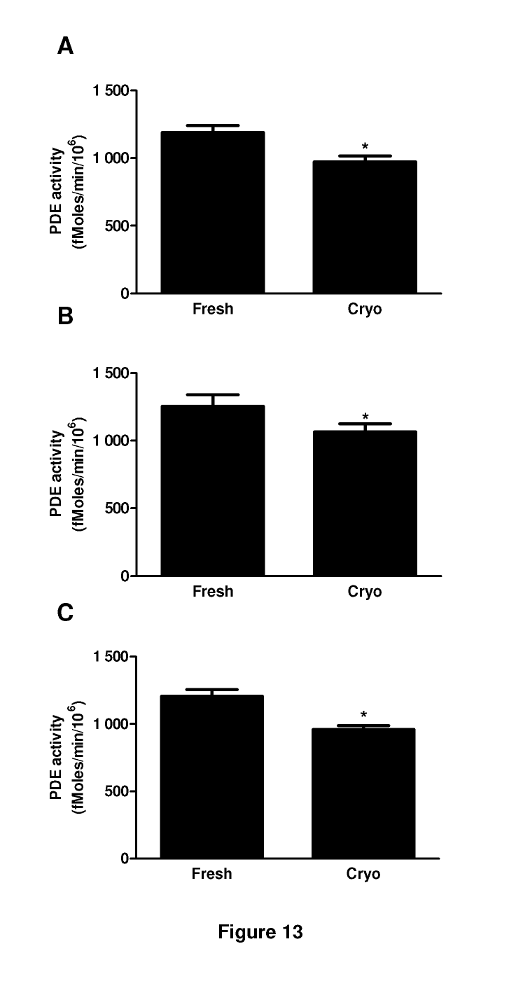

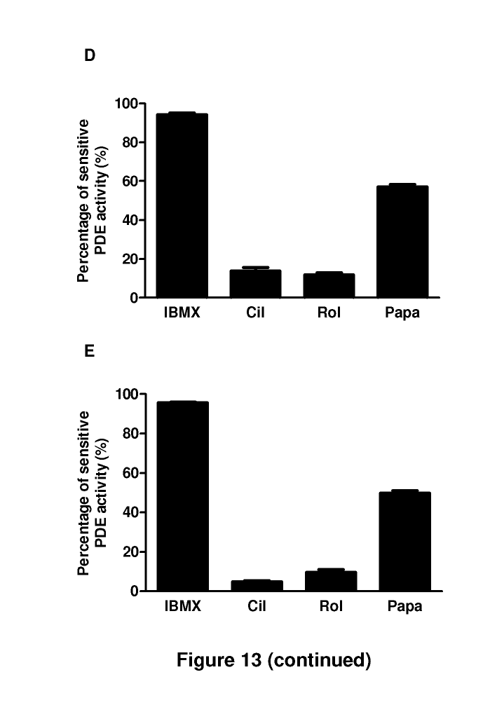

FIG. 13: There is a significant decreased in total PDE activity after cryopreservation. A closer look reveals that the percentage of papaverine-sensitive PDE activity decreased from 57.1%±1.3 in fresh spermatozoa to 49.8%±1.3 in cryopreserved spermatozoa. This decrease in papaverine-sensitive PDE activity can explain 75% of the total decrease in PDE activity, thereby supporting a major contribution of PDE10 in this decrease of PDE activity.

TABLE 2
Summary of PDE activity measured in bovine spermatozoa as in FIG.
14A, B and C.

| PDE activity | | | |
|---|---|---|---|
| (fMoles/min/×10⁶) ± SEM | Fresh | Cryo | Fold difference |
| All samples | 1202 ± 56 | 983 ± 45 | 1.22 |
| Per bull | 1252 ± 86 | 1064 ± 61 | 1.18 |
| Corrected per bull | 1206 ± 48 | 959 ± 29 | 1.26 |

TABLE 3
Summary of the contribution of different PDE inhibitors to the total PDE
activity measured in bovine spermatozoa.

| Percentage of sensitive | | |
|---|---|---|
| PDE activity | Fresh (A) | Cryo (B) |
| IBMX | 94 | 96 |
| Cilostamide | 14 | 5 |
| Rolipram | 12 | 10 |
| Papaverine | 57 | 50 |

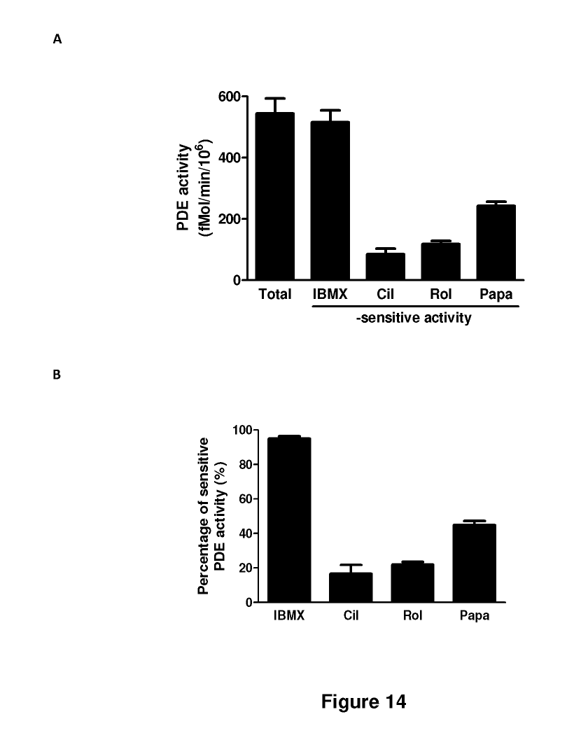

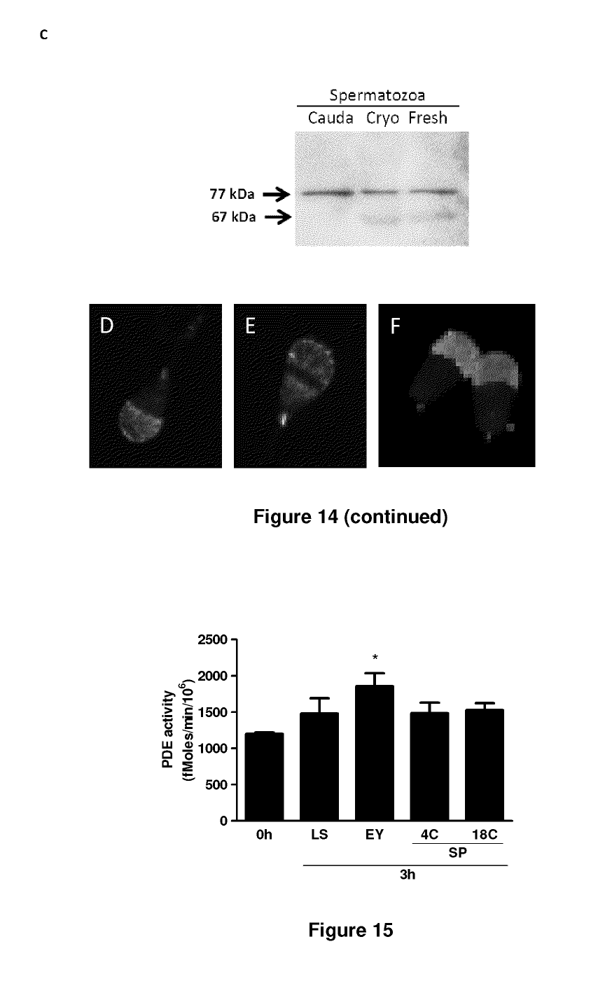

Table 2 and Table 3 summarize the data presented in FIG. 14. This figure demonstrates how the enzymatic assay is measuring a differential in PDE activity and that papaverine sensitive-PDE activity is mainly involved in this decrease.

FIG. 14: Epididymal spermatozoa possess a papaverine-sensitive PDE activity. The immunological pattern supports the functional presence of PDE10A with the presence of 77 kDa protein. The immunolabelling suggest a role in the fertilization process with the labelling in the acrosomal region. This figure is shows that the functional presence of PDE10A in bovine spermatozoa is acquired before ejaculation which supports the critical importance of its functional presence from cauda epididymis until ejaculation and post cryopreservation. This confirms that the PDE10 activity in the sperm does not originate from PDE10 in the seminal plasma which could have stuck to the spermatozoa after ejaculation. These data suggest that the PDE assay may be used in these epididymal spermatozoa to further discriminate the quality of the spermatozoa population such as in human. The therapeutic approach could also be used in epididymal of spermatozoa.

FIG. 15: A significant increase in PDE activity is measured after incubation in presence of egg yolk extender for 3 h supporting a deregulation of this enzyme activity while incubated in egg-yolk based extender. These data suggest that both total PDE activity and PDE10 activity rapidly increases after ejaculation and the addition of papaverine in the sperm sample would be a strategy to decrease PDE activity and further improve the quality of the spermatozoa. Table 4 summarizes the data presented in FIG. 15 and shows in more details the percentage of PDE10 in each treatment after 3 h of incubation.

TABLE 4
Percentage of family specific sensitive PDE activity measured in
different incubations conditions.

| | | 3 h | | | |
|---|---|---|---|---|---|
| | | Liquid | | | |
| | Fresh | semen | Egg yolk | 4° C. | 18° C. |
| Total PDE | 1200 ± | 1481 ± | 1856 ± 178 | 1483 ± 145 | 1527 ± 96 |
| activity ± | 18 | 207 | | | |
| SEM | | | | | |
| % IBMX- | 95% | 96% | 96% | 96% | 95% |
| Sens | | | | | |
| % Cil-Sens | 13% | 7% | 24% | 11% | 20% |
| (PDE3) | | | | | |
| % Rol-Sens | 11% | 11% | 18% | 12% | 9% |
| (PDE4) | | | | | |
| % Papa-Sens | 57% | 56% | 60% | 60% | 59% |
| (PDE10) | | | | | |

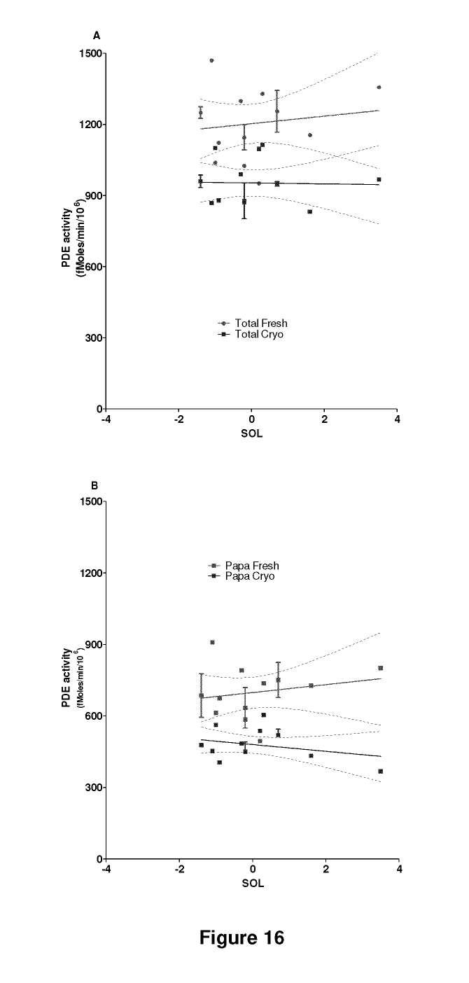

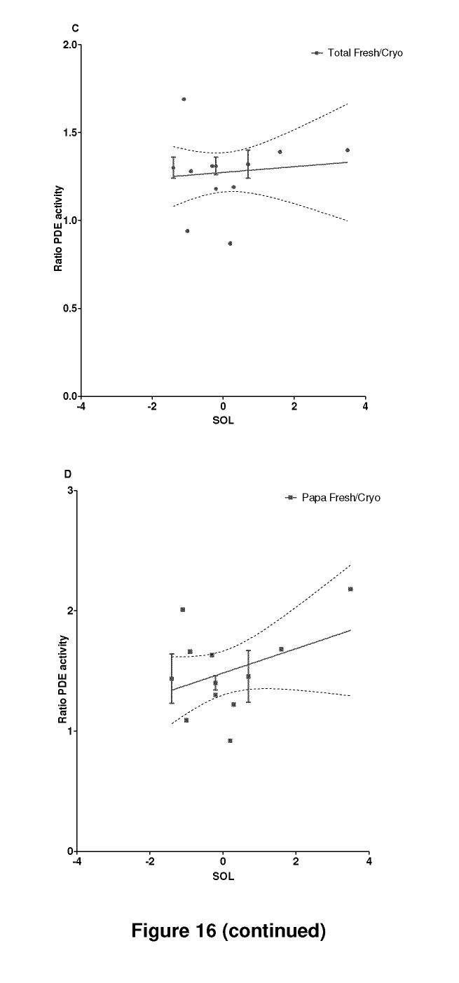

FIG. 16: There is a positive linear relationship between the fertility index of the bulls and the ratio of papaverine-sensitive PDE activity measured in freshly ejaculated spermatozoa over papaverine-sensitive PDE activity measured in the same ejaculated of spermatozoa but after cryopreservation. These data shows that a low ratio suggests a low fertility index while a higher ratio is be found in high fertility index bulls.

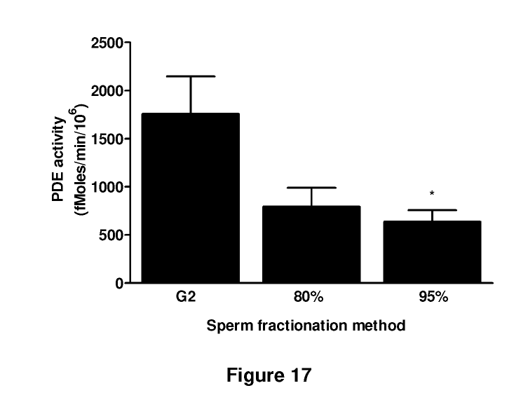

FIG. 17: PDE activity is measured in human spermatozoa after using 4 layers Percoll™ gradient (95%) fractionation method. G2 method is contaminated with other cell types in human as supported by the Table 5. The result shows that the 4 layers Percoll™ gradient 95% is appropriately isolating human spermatozoa and PDE10 is measured in human spermatozoa.

TABLE 5
Cell population analysis after three different sperm fractionation
methods

| | | Two-step | 4 layers |
|---|---|---|---|
| | G2 | Percoll ™ gradient | Percoll ™ gradient |
| | method | 40-80% | 95% |
| Rounds cells | +++ | + | − |
| Dead spermatozoa | ++ | + | − |
| Motile spermatozoa | + | ++ | +++ |

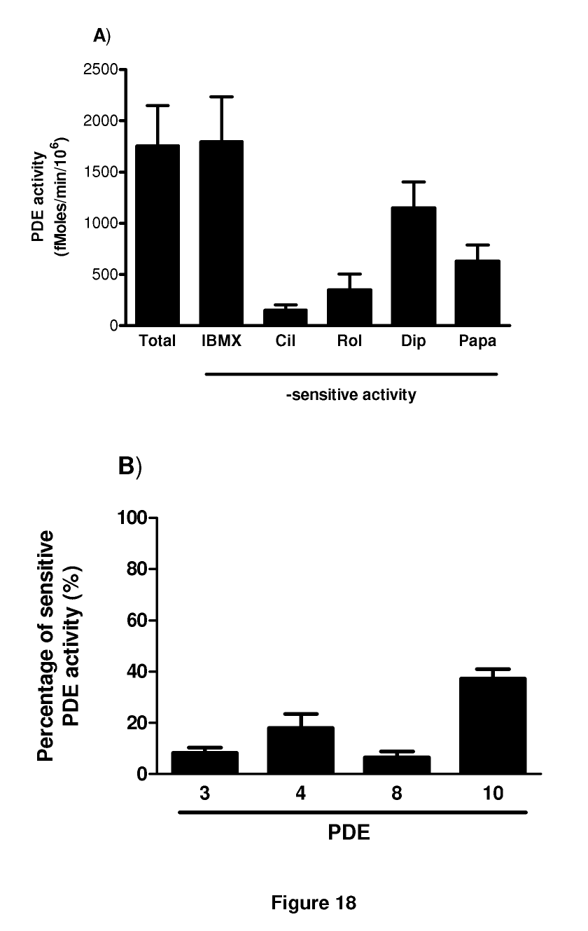

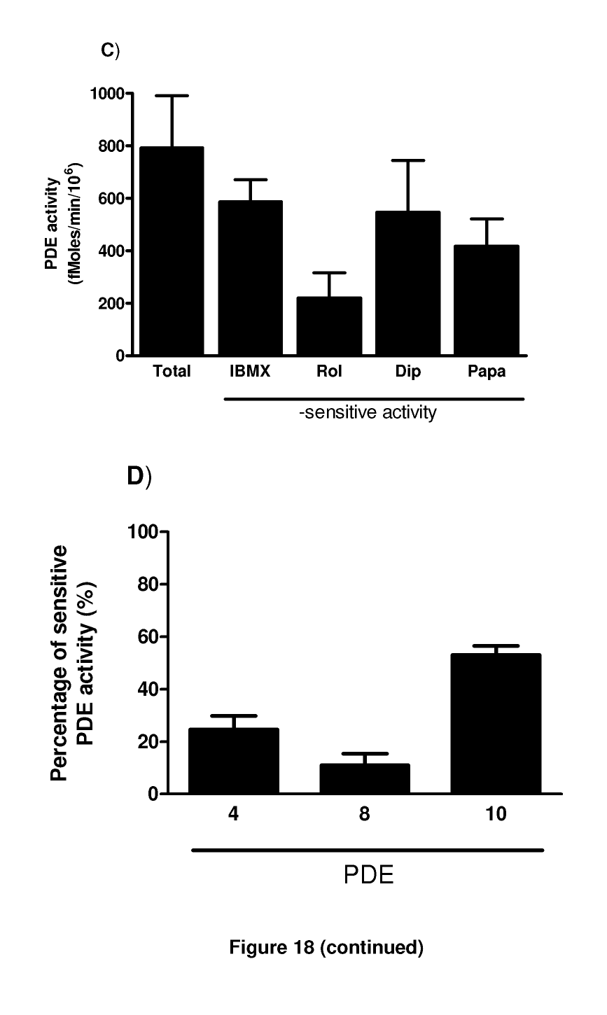

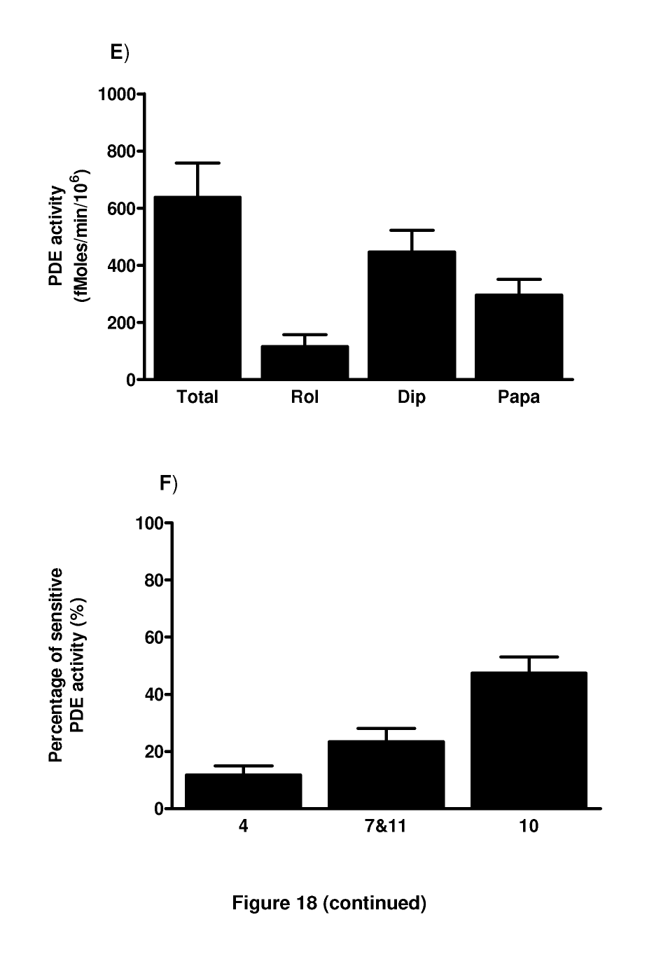

FIG. 18: PDE activity was measured in human ejaculate. The data shows papaverine-sensitive PDE activity in all three methods. The use of the 95% fractionation method directed in isolating motile spermatozoa resulted in more than 50% of cAMP-PDE activity measured as being papaverine-sensitive, which supports the functional presence of PDE10 in human spermatozoa, and the use of PDE assay in human.

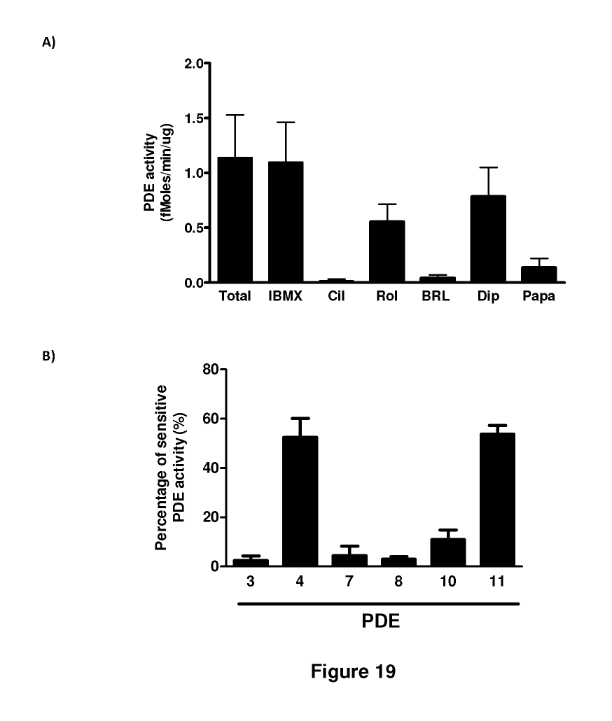

FIG. 19: PDE activity was measured in seminal plasma from a human ejaculate. A low percentage of papaverine-sensitive PDE activity was found but the high rolipram-sensitive PDE activity measured supports the functional presence of PDE4. Therefore, even if PDE10 activity is low in human seminal plasma, PDE10 activity in human spermatozoa is predominant.

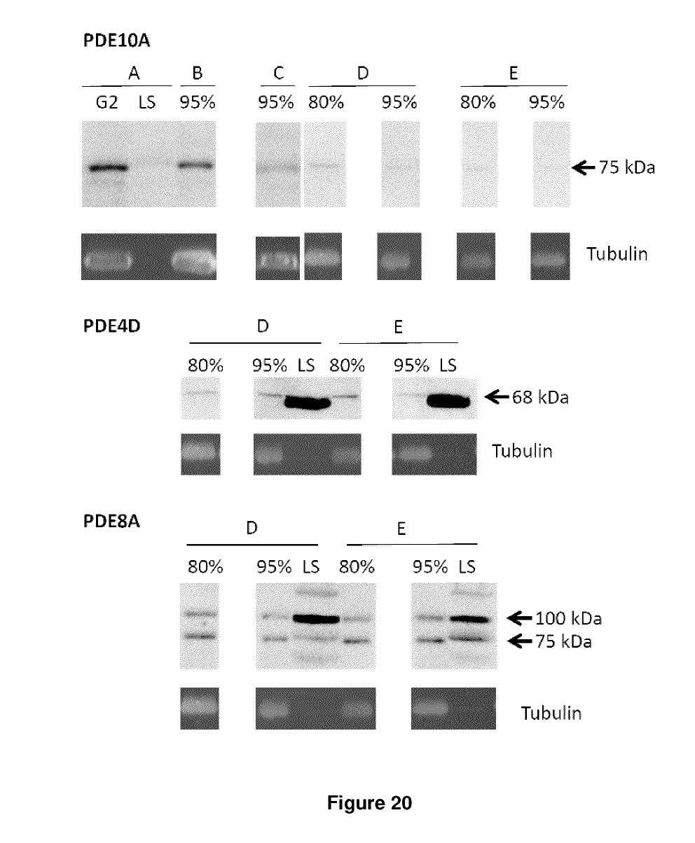

FIG. 20: Immunoblot of PDE10A, PDE4D and PDE11A supports the presence of PDE10A in human spermatozoa with a molecular weight of 75 kDa. PDE8A and PDE4D are also present.

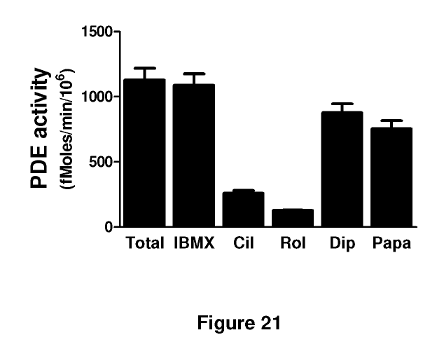

FIG. 21: This figure shows that papaverine-sensitive PDE activity efficiently inhibits more than 50% of 3′-5′ cAMP-PDE activity which correspond to PDE10. While using dipyridamol, a PDE inhibitor for PDE7, PDE8, PDE10 and PDE11 families, the proportion is increased by 10% which is suggested by its action on PDE8. Since IBMX-insensitive PDE activity is known to be associated to PDE8 family, it is not surprising to observe an increase of 10% since the percentage of IBMX-insensitive PDE activity is 8%. Then, dipyridamole is another PDE inhibitor that can be used for measuring PDE activity and PDE10-sensitive activity and in sperm sample to inhibit PDE10 activity in bovine spermatozoa. FIG. 18 thus supports the use of dipyridamole in human spermatozoa when measuring PDE activity and for addition in a sperm sample.

### Example 2

### Clinical Applications in Humans

A sperm ejaculate is obtained from a human donor. Once the ejaculate liquefied, spermatozoa are isolated on a Percoll™ gradient or by any other methods, and then the isolated spermatozoa are contacted with an aqueous solution comprising a PDE10 inhibitor such as papaverine. The aqueous solution comprising papaverine may be a medium such as TALP, or a semen extender, or a culture medium or buffer for liquid semen. In an alternative approach, the PDE10 inhibitor (e.g. papaverine) is added directly into the ejaculate after liquefaction. Exposure of the sperm with the PDE10 inhibitor can last for a few minutes or few hours before the sperm is used for IVF, for artificial insemination or for cryopreservation.

### Example 3

### Applications for the Dairy Cow Industry

A sperm ejaculate is harvested from a bull. The ejaculate then quickly receives (e.g. within 5 to 60 min) a dose (e.g. between 400 nm and 800 nm) of PDE inhibitor(s), preferably a PDE10 specific inhibitor such as papaverine. Exposure of the bovine sperm with the PDE10 inhibitor can last for a few minutes or few hours before the sperm is used for IVF, for artificial insemination or for cryopreservation.

In an alternative approach, the semen is processed for cryopreservation and the sperm is contacted with a PDE inhibitor (preferably a PDE10 specific inhibitor such as papaverine) that is present in a pre-freezing solution and/or present in an egg yolk based extender. Similarly, the sperm may be contacted with other type of medium or aqueous solutions, such as TALP, liquid semen culture media, or the like which have been supplemented with the PDE inhibitor.

In cases where the semen is already frozen, the semen is thaw and the sperm are quickly washed and placed in the presence of a PDE inhibitor specific to PDE10 such as papaverine, before being processed for IVF or artificial insemination.

Headings are included herein for reference and to aid in locating certain sections. These headings are not intended to limit the scope of the concepts described therein under, and these concepts may be applicable in other sections throughout the entire specification. Thus, the present invention is not intended to be limited to the embodiments shown herein but is to be accorded the widest scope consistent with the principles and novel features disclosed herein.

As used herein and in the appended claims, the singular forms “a”, “an”, and “the” include plural referents unless the context clearly indicates otherwise. Thus, for example, reference to “a compound” includes one or more of such compounds, and reference to “the method” includes reference to equivalent steps and methods known to those of ordinary skill in the art that could be modified or substituted for the methods described herein.

Unless otherwise indicated, all numbers expressing quantities of ingredients, reaction conditions, concentrations, properties, and so forth used in the specification and claims are to be understood as being modified in all instances by the term “about”. At the very least, each numerical parameter should at least be construed in light of the number of reported significant digits and by applying ordinary rounding techniques. Accordingly, unless indicated to the contrary, the numerical parameters set forth in the present specification and attached claims are approximations that may vary depending upon the properties sought to be obtained. Notwithstanding that the numerical ranges and parameters setting forth the broad scope of the embodiments are approximations, the numerical values set forth in the specific examples are reported as precisely as possible. Any numerical value, however, inherently contains certain errors resulting from variations in experiments, testing measurements, statistical analyses and so forth.

It is understood that the examples and embodiments described herein are for illustrative purposes only and that various modifications or changes in light thereof will be suggested to persons skilled in the art and are to be included within the present invention and scope of the appended claims.

1. A method for evaluating a mammalian sperm sample, the method comprising measuring sperm phosphodiesterase (PDE) enzymatic activity in said sperm sample, wherein said PDE activity is indicative of sperm ability to maintain its physiological functions during conservation.

2. The method of claim 1, wherein said PDE enzymatic activity is measured in isolated spermatozoa.

3. The method of claim 1, wherein measuring said PDE enzymatic activity comprises comparing sperm PDE enzymatic activity before and after freezing said sperm sample.

4. The method of claim 1, comprising measuring a ratio of PDE enzymatic activity before freezing over a PDE enzymatic activity after freezing, and wherein a ratio of about 1.5 or higher is predictive of a greater fertility than a ratio of about 1.0 or lower.

5. The method of claim 1, wherein said physiological functions are selected from the group consisting of: semen quality, semen fertility, sperm viability, sperm motility, sperm capacitation, and semen resistance to cryopreservation.

6. The method of claim 1, wherein said sperm phosphodiesterase (PDE) enzymatic activity is a PDE10 activity.

7. The method of claim 1, wherein said sperm PDE enzymatic activity is cAMP papaverine-sensitive.

8. The method of claim 1, wherein said mammalian sperm sample is selected from the group consisting of human sperm, bovine sperm, porcine sperm, equine sperm, dog sperm, cat sperm, rat sperm, mouse sperm, lion sperm, tiger sperm, elephant sperm.

9. A method for preserving a mammalian sperm sample, the method comprising contacting said sperm sample with an effective amount of a phosphodiesterase (PDE) inhibitor, wherein said inhibitor maintains sperm physiological functions during conservation.

10. The method of claim 9, wherein said PDE inhibitor is a PDE10 inhibitor.

11. The method of claim 9, wherein said PDE inhibitor is papaverine.

12. The method of claim 9, wherein the PDE inhibitor is present at a final concentration of about 50 nM to about 1000 nm.

13. The method of claim 9, wherein said PDE inhibitor is selected from the group consisting of dipyridamole, pyrazoloquinolines, MP10, 2-{4-[-pyridin-4-yl-1-(2,2,2-trifluoro-ethyl)-1H-pyrazol-3-yl]-phenoxymethyl}-quinoline succinic acid, and 1-(4-(2-(2-fluoroethoxy)ethoxy)-3-methoxybenzyl)-6,7-dimethoxyisoquinoline

14. The method of claim 9, wherein said sperm sample is contacted with said PDE inhibitor within 60 min post ejaculation.

15. The method of claims 9, for preserving said sperm sample before, during and/or after manipulation, dilution, freezing, thawing, cell-sorting, sex-sorting step; and/or before, during and/or after in vitro fertilization, artificial insemination, and/or cryopreservation processes.

16. A method for cryopreserving a mammalian sperm sample, the method comprising contacting said sperm sample with an effective amount of a phosphodiesterase (PDE) inhibitor before freezing.

17. The method of claim 16, wherein said PDE inhibitor is a PDE10 inhibitor.

18. The method of claim 16, wherein said PDE inhibitor is papaverine.

19. The method of claim 16, wherein said sperm sample is contacted with said PDE inhibitor within 60 min post ejaculation.

20. A semen extender for preserving a sperm sample, said extender comprising an effective amount of a phosphodiesterase (PDE) inhibitor for maintaining sperm physiological functions during conservation.

21. The semen extender of claim 20, wherein said PDE inhibitor inhibits a cAMP papaverine-sensitive sperm PDE enzymatic activity.

22. The semen extender of claim 20, wherein said PDE inhibitor is a PDE10 inhibitor.

23. The semen extender of claim 20, wherein said PDE inhibitor is papaverine.

24. The semen extender of claim 20, wherein the PDE inhibitor is present at a final concentration of about 50 nM to about 1000 nm.
